(19) United States
(12) Patent Application Publication
(10) Pub. No.: US 2020/0308228 A1
(43) Pub. Date: Oct. 1, 2020

(54) PEPTIDE INHIBITORS OF HCV NS3/4A PROTEASE COMPRISING NON-PROTEINOGENIC AMINO RESIDUES

(71) Applicant: King Abdulaziz University, Jeddah (SA)

(72) Inventors: Moustafa E. EL-ARABY, Jeddah (SA); Abdelsattar Mansour Omar, Jeddah (SA); Mahmoud Abdelkhalek El- Faky, Jeddah (SA); Sameh Hamdy Abdelmageed Soror, Jeddah (SA); Maan Talaat Khayat, Jeddah (SA); Hani Zakariah Asfour, Jeddah (SA); Faida Hassan Bamane, Jeddah (SA)

(73) Assignee: King Abdulaziz University, Jeddah (SA)

(21) Appl. No.: 16/371,610

(22) Filed: Apr. 1, 2019

(51) Int. Cl. C07K 7/08; A61K 45/06; A61K 38/10; A61P 31/14; G01N 33/576

(52) U.S. Cl. CPC C07K 7/08; A61K 45/06; G01N 33/576; A61P 31/14; A61K 38/10

(57) ABSTRACT

Peptide inhibitors of activation of hepatitis C virus (HCV) NS3 protease are disclosed. They are analogs of the activation peptide HCV NS4 of residues 21-33 of SEQ ID NO: 2 and contain non-proteinogenic amino acids. Competitive binding studies showed the peptide analogs bind HCV NS3 protease at the activation site.

### SEQUENCE LISTING

This application contains a Sequence Listing which has been submitted electronically in ASCII format and is hereby incorporated by reference in its entirety. Said ASCII copy, created on Mar. 8, 2019, is named 518026US_SL.txt and is 20,503 bytes in size

### STATEMENT OF FUNDING ACKNOWLEDGEMENT

This project was funded by the National Plan for Science, Technology and Innovation (MAARIFAH), King Abdulaziz City for Science and Technology, the Kingdom of Saudi Arabia; Award number 12-BIO3193-03.

### BACKGROUND OF THE INVENTION

### Field of the Disclosure

The present invention relates to peptides and peptide compositions comprising non-proteinogenic amino acids that inhibit the enzymatic activity of hepatitis C virus (HCV) NS3 protease. The peptides and peptide compositions are useful for the treatment of hepatitis C infection.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Hepatitis C virus (HCV) belongs to the hepaciviruses other pathogenic viruses from Flaviviridae which are a major global health problem. Around 71 million people are infected and it is the seventh cause of death in the world [Chhatwal et al. (2018) “Estimation of Hepatitis C Disease Burden and Budget Impact of Treatment Using Health Economic Modeling” Infect Dis Clin North Am, 32, 461-480; and Stanaway et al. (2016)]. Chronic hepatitis C infection in the Middle East and North Africa imposes a considerable burden on the health care systems due to higher infection rate by the virus than the rest of the world [Harfouche et al. (2017) “Hepatitis C virus viremic rate in the Middle East and North Africa: Systematic synthesis, meta-analyses, and meta-regressions” PloS one, 12, e0187177-e0187177]. For example, Egypt has an epidemic of hepatitis C infection as it has one-fifth of all chronic hepatitis C patients in the world [Kouyoumjian et al. (2018) “Characterizing hepatitis C virus epidemiology in Egypt: systematic reviews, meta-analyses, and meta-regressions” Scientific reports, 8, 1661-1661]. While the Kingdom of Saudi Arabia has a lower incidence of HCV infection than Egypt, about 0.4% to 1.1% of general population is infected with the virus causing a significant health issue in the Kingdom, in particular, among high-risk groups such as renal dialysis patients having infection rate of about 50% [Bawazir et al. (2017) “Hepatitis C virus genotypes in Saudi Arabia: a future prediction and laboratory profile” Virology journal, 14, 208-208]. It is noteworthy to point out that Saudi Arabia,

Egypt and other Arab countries share the predominance of Genotype 4 among the hepatitis C patients [Ghaderi-Zefrehi et al. (2016) “The Distribution of Hepatitis C Virus Genotypes in Middle Eastern Countries: A Systematic Review and Meta-Analysis” Hepatitis Monthly, 16, e40357-e40357].

From clinical point of view, HCV is a chronic infection that causes serious complications leading to death. In 2015, 400,000 deaths were attributed to complication from HCV infection such liver cirrhosis and hepatocellular carcinoma. Despite several drawbacks, the combined therapy of ribavirin/interferon has been the standard treatment for hepatitis C patients for decades. Recently, direct acting antiviral (DAA) drugs became available and revolutionized the treatment of HCV infection. DAA drugs are efficient in most patients, as the treatment duration is shorter and accompanied with significantly reduced adverse effects compared to previously used treatments [Afdhal et al. (2014) “Ledipasvir and sofosbuvir for previously treated HCV genotype 1 infection” N Engl J Med, 370, 1483-93]. Unfortunately, some patients do not respond to existing DAA drugs due to the emergence of drug resistant strains of the virus. The time consuming process of identifying candidate antiviral compounds and developing one or more into an effective antiviral drugs is costly and restrictive, and the outcome is unpredictable [Colpitts and Baumert (2016) “Addressing the Challenges of Hepatitis C Virus Resistance and Treatment Failure” Viruses (2016) 8 (8) 226; and Bartenschlager et al. (2018) “Critical challenges and emerging opportunities in hepatitis C virus research in an era of potent antiviral therapy: Considerations for scientists and funding agencies” Virus Res, 248, 53-62].

HCV belongs to Hepacivirus, one of the four genera of Flaviridae family of viruses that also includes Flavivirus, Pestivirus and Pegivirus [Simmonds et al. (2017) “ICTV virus taxonomy profile: Flaviviridae” Journal of General Virology, 98, 2-3; Richard et al. (2017) “AXL-dependent infection of human fetal endothelial cells distinguishes Zika virus from other pathogenic flaviviruses” Proceedings of the National Academy of Sciences (2017) 114(8), 2024-2029; and Guzman et al. (2018) “Characterization of three new insect-specific flaviviruses: their relationship to the mosquito-borne flavivirus pathogens” The American journal of tropical medicine and hygiene, 98, 410-419]. The family includes pathogens such as dengue virus, yellow fever virus, Japanese encephalitis virus, West Nile virus and Zika virus that cause worldwide morbidity and mortality (https:-www.cdc.gov/vhf/virus-families/flaviviridae.html). All Flaviviridae viruses have a single-stranded, non-segmented RNA genome that encodes a single chain, non-functional polyprotein [Jones et al. (2008) “Architects of assembly: roles of Flaviviridae non-structural proteins in virion morphogenesis” Nature reviews microbiology(2008) 6 (9), 699-708]. The viral polyprotein is processed into E1, E2 and C structural and p7, NS2, NS3, NS4A, NS4B, NS5A and NS5B functional proteins upon cleavage by viral and host proteases [Tanji et al. (1995) “Hepatitis C virus-encoded nonstructural protein NS4A has versatile functions in viral protein processing” Journal of virology, 69, 1575-1581; and Jones et al. (2008)]. In HCV, the multifunctional protein NS4A is a 54 amino acid hydrophobic peptide which is required for the activation of the protease and RNA-helicase domains of the NS3 polypeptide and the integration of the virus to the host cell endoplasmic reticulum [Ishido et al. (1998) “Complex formation of NS5B with NS3 and NS4A proteins of hepatitis C virus” Biochemical and biophysical research communications, 244, 35-40; Kim et al. (1995) “C-terminal domain of the hepatitis C virus NS3 protein contains an RNA helicase activity” Biochemical and biophysical research communications, 215, 160-166; and Wölk et al. (2000) “Subcellular localization, stability, and trans-cleavage competence of the hepatitis C virus NS3-NS4A complex expressed in tetracycline-regulated cell lines” Journal of virology, 74, 2293-2304]. In addition, NS3/NS4A plays important roles in neutralizing the host cell immune response to the viral invasion via cleavage and inactivation of CARDIF and TRIF, two critical sensing proteins that trigger antiviral responses [Li et al. (2005) “Immune evasion by hepatitis C virus NS3/NS4A protease-mediated cleavage of the Toll-like receptor 3 adaptor protein TRIF” Proceedings of the National Academy of Sciences, 102, 2992-2997; and Meylan et al. (2005) Cardif is an adaptor protein in the RIG-I antiviral pathway and is targeted by hepatitis C virus. Nature, 437, 1167].

The activation of NS3 protease is initiated by binding the hydrophobic N-terminus of NS4A of genotype 4 of SEQ ID NO: 2 to the protease domain between the A₀ (residues 4-10 of HCV genotype 4 of SEQ ID NO: 1) and A₁ (residues 32 to 38 of SEQ ID NO: 1) β-sheets at the N-terminal of the apoprotein to form an extended β-sheet [Kim et al. (1996); and Failla et al. “Both NS3 and NS4A are required for proteolytic processing of hepatitis C virus nonstructural proteins” (1994). Journal of virology, 68, 3753-3760]. The assembly and align the β-sheets of the NS3 together and reposition a-helix (Residues 13-22 of SEQ ID NO: 1). Structural studies indicate that NS4A optimize the geometry of the catalytic triad (His-57/Asp-81/Ser-128 of SEQ ID NO: 1 to reveal the catalytic activity of the protease [Love et al. (1998) “The conformation of hepatitis C virus NS3 proteinase with and without NS4A: a structural basis for the activation of the enzyme by its cofactor” Clinical and Diagnostic Virology, 10, 151-156]. Several studies reported that only the central hydrophobic region of NS4A, for example HCV NS4A of genotype 4 residues 22-31 of SEQ ID NO: 2 is sufficient for NS3 in vitro protease activation [Butkiewicz et al. (1996) “Enhancement of hepatitis C virus NS3 proteinase activity by association with NS4A-specific synthetic peptides: identification of sequence and critical residues of NS4A for the cofactor activity” Virology, 225, 328-338; Kim et al. (1996) “Crystal structure of the hepatitis C virus NS3 protease domain complexed with a synthetic NS4A cofactor peptide” Cell, 87, 343-355; Lin et al. (1995) “A central region in the hepatitis C virus NS4A protein allows formation of an active NS3-NS4A serine proteinase complex in vivo and in vitro” Journal of virology, 69, 4373-4380; Shimizu et al. (1996) “Identification of the sequence on NS4A required for enhanced cleavage of the NS5A/5B site by hepatitis C virus NS3 protease” Journal of Virology, 70, 127-132; Tomei et al. (1996) “A central hydrophobic domain of the hepatitis C virus NS4A protein is necessary and sufficient for the activation of the NS3 protease” Journal of General Virology, 77, 1065-1070; Yan et al. (1998) “Complex of NS3 protease and NS4A peptide of BK strain hepatitis C virus: a 2.2 A resolution structure in a hexagonal crystal form” Protein Sci, 7, 837-47; and U.S. patent application Ser. No. 10/319,402 (Joyce et al.); each incorporated herein by reference in their entirety]. Furthermore, serine and alanine mutation scanning studies revealed that the odd numbered hydrophobic residues Val-23, Ile-25, Gly-27, Val-29 and Leu-31 of SEQ ID NO: 2 are important to the enzyme activation process but not the even numbered residues because they are buried under the surface of the enzyme [Shimizu et al. (1996), Joyce et al. (2003)-incorporated herein by reference in its entirety]. The NS4A binding site was proposed as a target for allosteric NS3 inhibitors shortly after the identification of NS3/NS4A protease as a target for development of antiviral agent against HCV antiviral agents [Kim et al. (1996) and Shimizu et al. (1996)-each incorporated herein by reference in their entirety]. It was postulated that competitive ligands for the binding site of NS4A would alter the NS3 structure and the active site geometry leading to inactivation of the enzyme [Butkiewicz et al. (1996); Hamad et al. (2016) “The NS4A cofactor dependent enhancement of HCV NS3 protease activity correlates with a 4D geometrical measure of the catalytic triad region” PloS one, 11, e0168002; Kim et al. (1996); Shimizu et al. (1996)--each incorporated herein by reference in their entirety].

Shimizu et al. (1996) reported that replacing the positively charged Arg-28 of NS4A of SEQ ID NO: 2 with a neutral Gln (R28Q) produced an inhibitor of NS3 that bound to the

NS4A pocket. Their interpretation based on the observation that the NS3 inhibition could be reversed only by increasing the concentration of native NS4A, but not by increasing the substrate concentration.

Tomei et al. (1996) discloses that the central hydrophobic domain of the hepatitis C virus NS4a protein is necessary and required for the activation of the HCV NS3 protease. While the reference teaches the parent peptide as an activator of the NS3 protease, it does not disclose that variants of the peptide having non-proteinogenic amino acid such as the peptide of SEQ ID NO: 2 are inhibitors of the protease activity.

U.S. Pat. No. 6,939.854B2—incorporated herein by reference in its entirety discloses HCV NC3 protease inhibitors having the general formula:

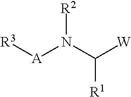

where 10 is a saturated alkyl group, R² is hydrogen, C1-C4 alkyl, aryl, aryl(C1-C4 alkyl) or C3-C6 cycloalkyl, R³, and W are selected from a boric acid derivative, an aldehyde, or a keto group. In some disclosed embodiments of the patent, A is a peptide comprising nonproteinogenic amino acid such as cyclohexylglycine, cyclohexylalanine, cyclopropylglycine, t-butylglycine, phenylglycine, and 3,3-diphenylalanine leading to a compound comprising a boric acid derivative, an aldehyde group, or a keto group.

US20030176689A1 - incorporated herein by reference in its entirety, discloses several peptide inhibitors of HCV NC3 protease based on the sequences of the NS4A/NSSB cleavage site, and on the sequence of the native activation NS4A₂₁₋₃₃ peptide of SEQ ID NO: 2. Some of the disclosed synthetic analogues of NS4A₂₁₋₃₃ of SEQ ID NO: 2 such as V23X, where X is L, t-L (tent-leucine), Pen (penacillamine), F or ChA (β-cyclohexylalanine) were tested for binding to NS3. V23F showed the highest binding affinity using frontal affinity chromatography in-line mass spectrometer (FAC-MS) analysis of equimolar mixture of synthetic peptide and NS3 enzyme. However, enzyme kinetic analysis based on cleavage of NS5A/5B peptide showed that the activity of NS3 increased in the presence V23F and most other mutant of NS4A at V23 activated less than that of wild-type NS4A. Other peptides comprising β-cyclohexylalanine (ChA), D-valine, and proteinogenic amino acids were able to inhibit NS3/NS4A protease activity were also disclosed.

Accordingly one object of the present disclosure is to provide one or more peptide, peptidomimetics, variants, homologs and/or compositions thereof that is capable of binding to HCV NC3 protease at the activation site and thereby inhibit the activation of the protease which is required for virus maturation.

### SUMMARY

A first aspect of the invention is directed to a peptide comprising an amino acid sequence having at least 60% sequence identity to Y₁GSX₁VX₂VGRX₃VLSGY₂ (SEQ ID NO: 5) and homologs thereof, and derivatives, analog, salt and/or solvate thereof, wherein at least one X₁, X₂, and X₃ is a non-proteinogenic amino acid, wherein the peptide inhibits the protease activity of NS3 protease of hepatitis C virus (HCV) of SEQ ID NO: 1 or a variant thereof having at least 60% sequence identity by binding to the binding site of the activation peptide of HCV NS4A of SEQ ID NO: 2 or variants thereof having amino acid sequence identity of at least 60% to SEQ ID NO: 2, and wherein Y₁ and Y₂ are independently selected from hydrogen, one or more charged amino acid residue, an organic moiety comprising ionizable group, and/or fluorescent moiety.

In a preferred embodiment, the HCV NS3 has at least 80% sequence identity to SEQ ID NO: 1.

In a more preferred embodiment, the HCV NS3 has at least 90% sequence identity to SEQ ID NO: 1.

In the most preferred embodiment, the HCV NS3 has 100% sequence identity to SEQ ID NO: 1.

In a preferred embodiment, the HCV NS4 has at least 80% sequence identity to SEQ ID NO: 2.

In a more preferred embodiment, the HCV NS4 has at least 90% sequence identity to SEQ ID NO: 2.

In the most preferred embodiment, the HCV NS4 has 100% sequence identity to SEQ

ID NO: 2.

In another preferred embodiment, the peptide has at least 80% amino acid sequence identity to SEQ ID NO: 5.

In another preferred embodiment, the non-proteinogenic amino acid is (S)-cyclohexylglycine (cG).

In another preferred embodiment, X₁, X₂, and X₃ are cG, I, and I, respectively.

In another preferred embodiment, X₁, X₂, and X₃ are V, cG, and I, respectively.

In another preferred embodiment, X₁, X₂, and X₃ are V, I, and cG, respectively.

In another preferred embodiment, the peptide has at least 70% sequence identity to an amino acid sequence selected from the group consisting of SEQ ID NO: 10, SEQ ID NO: 15, and SEQ IDNO: 20.

In a more preferred embodiment, the peptide is selected from the group consisting of SEQ ID NO: 10, SEQ ID NO: 15, and SEQ IDNO: 20.

In the most preferred embodiment, the peptide is SEQ ID NO: 20.

A second aspect of the invention is directed a pharmaceutical composition comprising one or more of the peptides of the invention.

In a preferred embodiment, the composition comprises one or more carriers and/or excipients.

In another preferred embodiment, the composition further comprises one or more additional antiviral compounds.

In another preferred embodiment, the variant peptide binds to the activation site of hepatitis C virus (HCV) NS3 protease.

In another preferred embodiment, the peptide has antiviral activity against the HCV.

In another preferred embodiment, the peptide has at least 70% sequence identity to an amino acid sequence selected from the group consisting of SEQ ID NO: 10, SEQ ID NO: 15, and SEQ IDNO: 20.

A third aspect of the invention is directed to a method of treating a subject infected with HCV comprising administering to the subject an effective amount of the pharmaceutical composition of invention.

A forth aspect of the invention is directed to a method of protecting a subject from getting infected with HCV comprising administering to the subject an effective amount of a pharmaceutical composition.

A fifth aspect of the invention is directed to a method of modifying a peptide selected from the group consisting of SEQ ID NO: 4, 9, 10, 14, 15, 19, and 20 to obtain a peptide or chemical compound having increased antiviral activity relative to the parent peptide, wherein the method comprises:

constructing a three dimensional model of a HCV NS3 having amino acid sequence identity of at least 90% to SEQ ID NO: 1 using the atomic coordinates of Protein Data Bank of accession number selected from the group consisting of 1NS3, 20B0, 208M, 2OBQ, and 20C 1,

docking a peptide selected from the group consisting of SEQ ID NO: SEQ ID NO: 4, 5, 9, 10, 14, and 15 to the three-dimensional model from (a),

modifying the structure of the docketed peptide to enhance the interactions between the resulting compound and the activator binding domain of an NS3 protease having at least 60% sequence identity to SEQ ID NO: 1,

synthesizing the compound having the modified structure, and

measuring the binding of the compound to an HCV NS3 protease having at 60% sequence identity to SEQ ID NO: 1,

wherein a peptide or compound having enhanced binding to the activation site relative to the parent peptide is identified as an antiviral compound.

In a preferred embodiment, a fluorescence assay method is used to measure the binding of the peptide or the compound to an HCV NS3 protease having at least 60% sequence identity to SEQ ID NO: 1.

In another preferred embodiment, a surface plasmon resonance binding assay is used to measure the binding of the peptide or the compound to an HCV NS3 protease having at least 60% sequence identity to SEQ ID NO: 1.

In another preferred embodiment, the identified peptide or compound contains a substitution of at least one amido nitrogen of at least one peptide bond with a -CR1R2 group wherein R1 and R2 are independently hydrogen, optionally substituted alkyl, optionally substituted cycloalkyl, optionally substituted aryl, optionally substituted heterocyclic, or optionally substituted heteroaryl.

In a more preferred embodiment, —CR₁R₂ is —CH₂.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows SDS-PADE of purified NS3 of SEQ ID NO: 3.

FIG. 2 shows (A) DSLS Spectra of HCV NS3 genotype 4a of SEQ ID NO: 3 without NS4A (left) and after mixing with KK-GSVVIVGRIVLSG-KK of (SEQ ID NO: 4) and comprising the native NS4A₂₁₋₃₃ of SEQ ID NO: 2 (right) for 2 h at room temperature and shaking.

FIG. 3A shows changes in NS3 stability expressed as differences in the aggregation temperature (ΔT_{agg})±SEM between NS3 of SEQ ID NO: 3 (15 μM) mixed with NS4A of SEQ ID NO: 4 or analogues thereof of Pep-1, SEQ ID NO: 6 to Pep-15 of SEQ ID NO: 20, and NS3 of SEQ ID NO: 3 for 2 h at 25 ° C. The bottom bar is 1:1 molar ratio of pepeptide:NS3 of SEQ ID NO: 3 and the top bar is 2:1 molar ratio of pepeptide:NS3 of SEQ ID NO: 3.

FIG. 3B shows aggregation of the NS3 protein under thermal denaturation (DSLS measurement). NS3 of SEQ ID NO: 3 alone (left) and a mixture of 15 μM NS3 of SEQ ID NO: 3 and 30 μM Pep-15 of SEQ ID NO: 20 (right).

FIG. 4 shows fluorescence anisotropy plot of a mixture FITC-NS4A of SEQ ID NO: 4/NS3 of SEQ ID NO: 3 (1.8 μM) against varied concentrations of Pep-15 of SEQ ID NO: 20.

FIG. 5 shows the displacement of the bound fluorescent labeled peptide (SEQ ID NO: 25) to NS3 of SEQ ID NO: 3 by the peptide of SEQ ID NO: 20. The plot and the dissociation constant were calculated using the “Single Binding Site Model” impeded in GraphPad Prism 7 software.

FIG. 6 shows NS3 of SEQ ID NO: 3 (1.8 μM) catalytic activities as measured by the change of fluorescence of the peptide substrate of SEQ ID NO: 22 in the presence and absence 0.1 μM of the peptide of SEQ ID NO: 25 comprising the activation sequence of NS4A, and in the presence and absence of Pep-15 of SEQ ID NO: 20 (0.001 to 50 μM).

FIG. 7A shows the crystal structure of NS3 of SEQ ID NO: 1 in light colored bound to NS4A polypeptide of SEQ ID NO: 2 (PDB Code: 1NS3) darker colored. The two β-sheets intercalates NS4A (A0 and A1 ) are the darkest color.

FIG. 7B shows interactions of NS4A of SEQ ID NO: 2 core residues with A1 sheet of NS3 of SEQ ID NO: 1.

FIG. 8 shows the planar core part of NS4A of SEQ ID NO: 2 with a glycine turn extending along residues Val-26, Gly27 and Arg-28.

### DETAILED DESCRIPTION

Embodiments of the present disclosure will now be described more fully hereinafter with reference to the accompanying drawings, in which some, but not all embodiments of the disclosure are shown. The present disclosure will be better understood with reference to the following definitions.

All publications mentioned herein are incorporated herein by reference in full for the purpose of describing and disclosing the methodologies, which are described in the publications, which might be used in connection with the description herein. Nothing herein is to be construed as an admission that the inventors are not entitled to antedate such disclosure by virtue of prior disclosure. Also, the use of “or” means “and/or” unless stated otherwise. Similarly, “comprise,” “comprises,” “comprising” “include,” “includes,” and “including” are interchangeable and not intended to be limiting.

As used herein, the term “compound” is intended to refer to a chemical entity, whether in a solid, liquid or gaseous phase, and whether in a crude mixture or purified and isolated.

As used herein, the term “salt” refers to derivatives of the disclosed compounds, monomers or polymers wherein the parent compound is modified by making acid or base salts thereof. Exemplary salts include, but are not limited to, mineral or organic acid salts of basic groups such as amines, and alkali or organic salts of acidic groups such as carboxylic acids. The salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally non-aqueous media like ether, ethyl acetate, ethanol, isopropanol, or acetonitrile are preferred.

As used herein, the term “about” refers to an approximate number within 20% of a stated value, preferably within 15% of a stated value, more preferably within 10% of a stated value, and most preferably within 5% of a stated value. For example, if a stated value is about8.0, the value may vary in the range of 8±1.6, ±1.0, ±0.8, ±0.5, ±0.4, ±0.3, ±0.2, or ±0.1.

As used herein, the term “alkyl” unless otherwise specified refers to both branched and straight chain saturated aliphatic primary, secondary, and/or tertiary hydrocarbons of typically C₁ to C₁₀, and specifically includes, but is not limited to, methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. As used herein, the term optionally includes substituted alkyl groups. Exemplary moieties with which the alkyl group can be substituted may be selected from the group including, but not limited to, hydroxyl, alkoxy, aryloxy, or combination thereof.

As used herein, the term “substituted” refers to at least one hydrogen atom that is replaced with a non-hydrogen group, provided that normal valences are maintained and that the substitution results in a stable compound. When a substituent is noted as “optionally substituted”, the substituents are selected from the exemplary group including, but not limited to, halo, hydroxyl, alkoxy, oxo, alkanoyl, aryloxy, alkanoyloxy, amino, alkylamino, arylamino, arylalkylamino, disubstituted amines (e.g. in which the two amino substituents are selected from the exemplary group including, but not limited to, alkyl, aryl or arylalkyl), alkanylamino, aroylamino, aralkanoylamino, substituted alkanoylamino, substituted arylamino, substituted aralkanoylamino, thiol, alkylation, arylthio, arylalkylthio, alkylthiono, arylthiono, aryalkylthiono, alkylsulfonyl, arylsulfonyl, arylalkylsulfonyl, sulfonamide (e.g. —SO₂NH₂), substituted sulfonamide, nitro, cyano, carboxy, carbamyl (e.g. —CONH₂), substituted carbamyl (e.g. —CONHalkyl, —CONHaryl, —CONHarylalkyl or cases where there are two substituents on one nitrogen from alkyl, aryl, or alkylalkyl), alkoxycarbonyl, aryl, substituted aryl, guanidine, heterocyclyl (e.g. indolyl, imidazoyl, furyl, thienyl, thiazolyl, pyrrolidyl, pyridyl, pyrimidiyl, pyrrolidinyl, piperidinyl, morpholinyl, piperazinyl, homopiperazinyl and the like), substituted heterocyclyl and mixtures thereof and the like.

As used herein, the term “cycloalkyl” refers to cyclized alkyl groups. Exemplary cycloalkyl groups include, but are not limited to, cyclopropyl, cyclobutyl, cyclopentyl, cyclohexyl, norbornyl, and adamantyl. Branched cycloalkyl groups such as exemplary 1-methylcyclopropyl and 2-methylcyclopropyl groups are included in the definition of cycloalkyl as used in the present disclosure.

As used herein, the term “aryl” unless otherwise specified refers to functional groups or substituents derived from an aromatic ring including, but not limited to, phenyl, biphenyl, naphthyl, thienyl, and indolyl. As used herein, the term optionally includes both substituted and unsubstituted moieties. Exemplary moieties with which the aryl group can be substituted may be selected from the group including, but not limited to, hydroxyl, amino, alkylamino, arylamino, alkoxy, aryloxy, nitro, cyano, sulfonic acid, sulfate, phosphonic acid, phosphate or phosphonate or mixtures thereof. The substituted moiety may be either protected or unprotected as necessary, and as known to those skilled in the art.

As used herein, the term “alcohol” unless otherwise specified refers to a chemical compound having an alkyl group bonded to a hydroxyl group. Many alcohols are known in the art including, but not limited to, methanol, ethanol, propanol, isopropanol, butanol, isobutanol and t-butanol, as well as pentanol, hexanol, heptanol and isomers thereof. Since the alkyl group may be substituted with one or more hydroxyl group, the term “alcohol” includes diols, triol, and sugar alcohols such as, but not limited to, ethylene glycol, propylene glycol, glycerol, and polyol.

As used herein a “polymer” or “polymeric resin” refers to a large molecule or macromolecule, of many repeating subunits and/or substances composed of macromolecules. As used herein a “monomer” refers to a molecule or compound that may bind chemically to other molecules to form a polymer. As used herein the term “repeat unit” or “repeating unit” refers to a part of the polymer or resin whose repetition would produce the complete polymer chain (excluding the end groups) by linking the repeating units together successively along the chain. The method by which monomers combine end to end to form a polymer is referred to herein as “polymerization” or “polycondensation”, monomers are molecules which can undergo polymerization, thereby contributing constitutional repeating units to the structures of a macromolecule or polymer. As used herein “resin” or “polymeric resin” refers to a solid or highly viscous substance or polymeric macromolecule containing polymers, preferably with reactive groups. As used herein a “copolymer” refers to a polymer derived from more than one species of monomer and are obtained by “copolymerization” of more than one species of monomer. Copolymers obtained by copolymerization of two monomer species may be termed bipolymers, those obtained from three monomers may be termed terpolymers and those obtained from four monomers may be termed quarterpolymers, etc. As used herein, “cross-linking”, “cross-linked” or a “cross-link” refers to polymers and resins containing branches that connect polymer chains via bonds that link one polymer chain to another.

As used herein, the term “biopolymer” referrers to biological molecules such as peptide, polypeptides, proteins, RNA, and DNA. Polypeptide and proteins comprises the 20 proteinogenic L-amino acid encoded by nucleic acids. They are glycine (Gly or G), Alanine (Ala or A), valine (Val or V), leucine (Leu or L), isoleucine (Ile, I), serine (Ser or S), threonine (Thr or T), cysteine (Cys or C), methionine (Met or M), proline (Pro or P), aspartic acid (Asp or D), asparagine (Asn or N), glutamic acid (Glu or E), glutamine (Gln or Q), lysine (Lys or K), arginine (Arg or R), histadine (His or H), phenylalanine (Phe or F), tyrosine (Tyr or Y), and tryptophan (Trp or W). As used herein, the term “L-amino acid” is identified by their names, single letter designation or three letters designation only. For example, Tyr or Y means L-tyrosine. On the other hand, the D-enantioners of said amino acids may have the same designation except that the notation is preceded by the letter D. For example, D-tyrosine is referred to as D-Tyr. The L and D convention for ammo acid configuration refers not to the optical activity of the amino acid itself, but rather to the absolute configuration of the amino acid. L-amino acids has the same absolute configuration as the levorotatory L-glyceraldehyde, whereas D-amino acid has the same absolute configuration dextrorotatory D-glyceraldehyde. Alternatively, (S) and (R) designators are used to indicate the absolute configuration at a chiral atom using a specific set rules which are found in any introductory Organic Chemistry text book [see for example; “Organic Chemistry” by Morrison and Boyd, 3^{rd} Ed, (1973) Chapter 4, section 4.15 at page 130]. Almost all amino acids in proteins have the S-configuration at the α-carbon, with only cysteine having R-configuration and glycine non-chiral. Cysteine has its side chain in the same geometric position as the other amino acids, but the R/S designation is reversed because sulfur has higher atomic number than that of the carboxyl oxygen given the side chain a higher priority than the carboxyl group. DNA and RNA are poly-2′-deoxynulcotide and polynucleotide, respectively, of adenine (A), guanine (G), thymidine (T, DNA only), uridine (U, RNA only), and cytidine (C).

As used herein, the term “non-proteinogenic amino acid refers to an organic compound or moiety comprising an amino group and acidic group that is not encoded by a nucleic acid codon. Examples of non-proteinogenic amino acids include but not limited to phenylglycine, 3-cyclopropyl alanine, 3-cyclohexylalanine, 3-fluoralanine, hexylglycine, halogenated phenylalanine such as, but not limited to 4-fluorophenyl alanine, 3,4-difluorphenylalanine, L- and D-hydroxyprolein, perfluorphenylalanine, alkyl histidine, o-, m-, p-aminobenzoic acid, 2-aminonaphthoic acid and isomers thereof, halogenated histidine, 3-trazoloalanine, 3-tetrazoloalanine, homocysteine, homoisoleucine [2-amino-4-methyhexanoic acid] and the like. All stereoisomer including the stereoisomers of proteinogenic amino acid are considered non-proteinogenic amino acids.

As used herein the phrase “sequence identity” describes the “%” identity between two amino acid sequences. The two amino acid sequences aligned in such a way to maximize the matching of amino acid residues in the two sequences. The sum of the identical amino acid in a sequence divided by the total number of amino acid residues in a peptide is the percentage of sequence identity. For example, two 10 amino acid residues peptides that differ by one amino acid residue are 90% identical.

As used herein, the term “solvate” refers to a physical association of a compound, monomer or polymer of this disclosure with one or more solvent molecules, whether organic or inorganic. This physical association includes hydrogen bonding. In certain instances, the solvate will be capable of isolation, for example when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. Solvate encompasses both solution phase and isolable solvates. Exemplary solvates include, but are not limited to, hydrates, ethanolates, methanolates, isopropanolates and mixtures thereof. Methods of solvation are generally known to those of ordinary skill in the art.

As used herein, the term “activation site” refers to the activation site of the NS3 protease of SEQ ID NO: 1 wherein the peptide of NS4 of SEQ ID NO: 2 binds and activates the protease activity. The activation site is different from the site where the substrate binds to an enzyme.

A first aspect of the invention is directed to a peptide comprising an amino acid sequence having at least 60% sequence identity to Y₁GSX₁VX₂VGRX₃VLSGY₂ (SEQ ID NO: 5) and homologues thereof and derivatives, salts and/or solvates thereof, wherein at least one of X₁, X₂, and X₃ is a non-proteinogenic amino acid, wherein the peptide inhibits the protease activity of NS3 protease of hepatitis C virus of SEQ ID NO: 1 or a variant thereof having at least 60% sequence identity by binding to the binding site of the activation peptide NS4A of SEQ ID NO: 2 or variants thereof having amino acid sequence identity of at least 60% to SEQ ID NO: 2 or fragment thereof, and wherein Y₁ and Y₂ are independently selected from hydrogen, one or more amino acid residue, preferably one or more charged amino acid, an organic moiety comprising ionizable group(s), and/or fluorescent moiety. The peptide of the invention has at least at least 50%, preferably at least 60%, more preferably 70%, even more preferably 80%, and most preferably 90% amino acid sequence identity to SEQ ID NO:

5.

As used herein, the terms “HCV NS3 protease” or “NS3 protease” are used interchangeably and have the same meaning, and have an amino acid sequence in the range of 60% to 100% sequence identity to SEQ ID NO: 1. In some embodiment the sequence identity is at least 60%, at least 65%, at least 70%, at least 75%, at least 80%, at least 85%, at least 90%, at least 95% and at least 100%.

As used here in the terms “HCV N54”, “N54” or activation peptide are used interchangeably refereeing to a peptide having an amino acid sequence in the range of 60% to 100%. In some embodiment, the peptide is at least 60%, at least 65%, at least 70%, at least 75%, at least 80%, at least 85%, at least 90%, at least 95% and at least 100%. Since the peptide corresponding to residues 21 to 33 of SEQ ID NO: 2 comprises mostly hydrophobic amino acids, an organic moiety containing one or more charged groups at N- and/or C-terminus aids the solubility of the peptide in aqueous solution, in particular in a pH range of 6.0 to 9.0. In some preferred embodiments, the charged organic moiety of Y₁ and Y₂ is one or more amino acid residues comprising one or more charged amino acids selected from the group consisting of lysine, arginine, glutamic acid, and aspartic acid with the proviso that the peptide has one or more net charges in the pH range of 6.0 to 9.0. In a more preferred embodiments, Y₁=Y₂ is one, two, three or even more aspartic acid, glutamic acid, lysine, arginine residue or combination thereof. In a particularly preferred, Y₁=Y₂ is selected from dilysine, diarginine, lysin-arginine, and arginine-lysine. Also, Y₁ and Y₂ may be other moiety comprising an ionizable group such as but not limited to phosphate, sulfonic acid, 1-aminoethane sulfonic acid, 2-aminoethylphosphate, 2-aminoethyldiphosphate, 2-aminoethylphosphonic acid and the like.

Any organic compound comprising an amino group and an acidic group such as COOH, SO₃H, or PO₃H, and the like are considered non-proteinogenic amino acids and may be used in making the peptide of the invention of SEQ ID NO: 5, in particular, as long as the resulting peptide binds to the activation site of HCV NS3 protease and inhibits the formation of the active form of the enzyme.

In some preferred embodiments, the non-proteinogenic amino acid has the amino acid formula I:

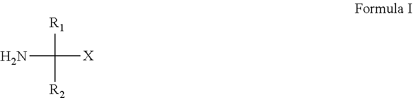

where R₁ and R₂ are independently hydrogen, optionally substituted alkyl, optionally substituted cycloalkyl, optionally substituted heterocycloalkyl, optionally substituted aryl, and optionally substituted heteroaryl, or a part of a three, four, five, six, seven or eight membered ring. The alkyl group may be saturated or unsaturated alkyl group. Examples of saturated alkyl groups such as but not limited to include optionally substituted or unsubstituted methyl, ethyl, n-propyl, isopropyl, cyclopropyl, n-butyl, s-butyl, t-butyl, cyclobutyl; n-pentyl and isomers thereof, cyclopentyl, n-hexyl and isomers thereof, and cyclohexyl with the proviso that the amino acid is not a proteinogenic amino acid. Formula I may have the (S) or (R) configuration, preferably the (S)-configuration. In a preferred embodiment, the nonproteinogenic amino acid is (S)- or (R)-2-amino-4-methyhexanoic acid, more preferably the (S)-enantiomer (also known as hI or (S)-homoisoleucine), and isomers thereof. The optionally substituted or unsubstituted aryl include but not limited to phenyl and naphthyl groups. Examples of amino acid where R₁ and R₂ are part of a ring include, but not limited to 1-amino-1-carboxylcyclopropane, 1-amino-l-carboxylcyclobutane, 1-amino-1-carboxylcyclohexane, 1-amino-1-carboxylcycloheptane, and 1-amino-1-carboxylcyclooctane as well as their isomers.

In some preferred embodiments, the non-proteinogenic amino acid (X) is alkyl substituted glycine at C2 wherein the alkyl group is optionally substituted cycloalkyl group such as but not limited to cyclopropyl, cyclobutyl, cyclopentl, cyclohexyl, cycloheptyl, and cycloctyl. The non-prteinogenic amino acid may have either the (S)- or (R)-configuration. In a particularly preferred embodiment, the non-proteinogenic amino acid is (S)-cyclohexylglycine (cG).

In a preferred embodiment of SEQ ID NO: 5, X₁, X₂, and X₃ are cG, I, and I, respectively. In another preferred embodiment of SEQ ID NO: 5, X₁, X₂, and X₃ are V, cG, and I, respectively. In yet another preferred embodiment of SEQ ID NO: 5, X₁, X₂, and X₃ are V, I, and cG, respectively.

In some other preferred embodiments of SEQ ID NO: 5, X₁, X₂, and X₃ are hI, I, and I, respectively. In another preferred embodiment of SEQ ID NO: 5, X₁, X₂, and X₃ are V, hI, and I, respectively. In yet another preferred embodiment of SEQ ID NO: 5, X₁, X₂, and X₃ are V, I, and hI, respectively.

### Peptide Synthesis:

The peptides and their analogs of the invention may be obtained by well-known chemical synthetic methods. Peptides comprising up to 5, 10, 15, 20, 25, 30, 35, 40 amino acid residues may be prepared by chemical synthesis. The advantage of the chemical synthesis is that any chemical compound having an amino group and COOH, SO₃H, or PO₃H group may be incorporated into any peptide. The chemical methods for peptide synthesis are well-known in the art and taught in many standard text books such as Creighton, T. E. [Proteins; Structures and molecular properties, second edition (1993) W. H. Freeman and Company, incorporated herein by reference]. The peptide may be synthesized in solution or on a solid support. Solution methods may be used to prepare short peptides (less than 5-6 amino acid residue) by coupling two appropriately protected amino acids, one of which has a free amino group and the other has a free carboxyl group using a coupling reagent including but not limited to dicyclohexylcarbodiimide (DCC) in any suitable solvent such as methylene chloride to produce a dipeptide with protected carboxyl and amino termini. One of the termini is selectively unprotected and the resulting peptide is coupled to another amino acid and the process is repeated until the desired sequence is made. Once the peptide is made, all the protecting groups can be removed by well-known methods in the art such as acid treatment, catalytic hydrogenation, and mild base hydrolysis. In the 1960's, Bruce Merrifield developed the method of solid support synthesis of peptides which became the method of choice of making peptides of up to 60 amino acid in length and even longer. Several reviews of the method and its application are described in details in the prior art, see for example Merrifield, B. [“Solid phase synthesis” Science (1986)232, 241-247, incorporated herein by reference in its entirety] and Sheppard, R. C. [“Modern Methods of solid phase peptide synthesis” Science Tools (1986) 33, 9-16, incorporated herein by reference in its entirety]. The method utilizes polymeric resins functionalized with amino groups or hydroxyl groups to which a properly protected amino acid is attached followed by deprotecting an amino or carboxyl group. The resulting amino or carboxyl group can be coupled to another amino acid residue using a coupling reagent such as DCC. The process is fully automated and can produce peptides efficiently especially in the range 4 to 60 amino acid residues in large quantities. Once the peptide is assembled on the solid phase, the peptide is liberated from the solid phase by hydrogen fluoride treatment to produce the peptide without any protecting groups. All proteinogenic amino acid and their stereoisomer as well as many non-proteinogenic amino acids properly protected and other reagents for use in automated peptide synthesis systems are commercially available. The synthesis of many non-proteinogenic amino acids are described in the prior art and can be their N-, and C-termini can be protected by well-known methods in the art. The structure of the resulting peptide can be verified by amino acid composition analysis and spectroscopic methods such as NMR spectroscopy and mass spectrometry.

Pharmaceutical Composition:

A second aspect of the invention is related to a pharmaceutical composition comprising one or more peptides having at least 55%, 60%, 65%, 70%, 75%, 80%, 85%, 90%, or 95% sequence identity to SEQ ID NO: 10, 15, and 20 that inhibits the activation of the HCV NS3 protease.

As used herein, a “composition” or a “pharmaceutical composition” refers to a mixture of the active ingredient with other chemical components, such as pharmaceutically acceptable carriers and excipients. One purpose of a composition is to facilitate administration of the peptides of the invention. Pharmaceutical compositions of the present disclosure may be manufactured by processes well-known in the art, e.g., by means of conventional mixing, dissolving, granulating, dragee-making, levigating, emulsifying, encapsulating, entrapping or lyophilizing processes. Depending on the intended mode of administration (oral, parenteral, or topical), the composition can be in the form of solid, semi-solid or liquid dosage forms, such as tablets, suppositories, pills, capsules, powders, liquids, or suspensions, preferably in unit dosage form suitable for single administration of a precise dosage.

The term “active ingredient”, as used herein, refers to an ingredient in the composition that is biologically active, for example, the peptides of SEQ ID NO: 10, 15, and 20 or homologs thereof which may comprise a salt, a solvate, or any mixtures thereof.

A preferred embodiment, the pharmaceutical composition comprises one or more of the peptide of the invention in the range of 5% to 100%, more preferably in the range of 50% to 90%, even more preferably in the range of 60% to 85%, and most preferably 75% to 80% based on the total weight of composition.

In some embodiments, the pharmaceutical composition comprises up to 0.1 wt. %, 1 wt. %, 5 wt. %, or 10 wt. % of the total weight of a pharmaceutically acceptable salt other than the peptide salt. In some embodiments, the pharmaceutical composition comprises up to 0.1 wt.%, 0.5 wt.%, 1.0 wt.%, 2.0 wt.%, 3.0 wt.%, 4.0 wt.%, 5.0 wt.%, or 10.0 wt.% of a pharmaceutically acceptable solvate. Preferably, the pharmaceutical composition may further comprise pharmaceutically acceptable binders, such as sucrose, lactose, glucose, fructose, galactose, mannitol, xylitol, and pharmaceutically acceptable excipients such as calcium carbonate and calcium phosphate.

Since small peptides in the range of 2 to 20 amino acids in length have short half-life time in biological environment, the peptide may be conjugated to a protein or polymer. In particular, polyethylene glycol (PEG) has many known advantages in formulating peptides and proteins pharmaceuticals. Not only conjugation of peptides and protein to PEG increases the half-life time of the peptide or protein in biological system, but also minimizes the immune response to the peptide or protein. The peptides of the invention may be conjugated by well-known methods in the art to an appropriate PEG preparation.

In a preferred embodiment, the pharmaceutical composition comprises the peptide of the invention at a concentration in the range of 1.0 μM to 100 mM. In another preferred embodiment, the pharmaceutical composition comprises one or more carriers and/or excipients selected from the group consisting of a buffer, an inorganic salt, a fatty acid, a vegetable oil, a synthetic fatty ester, a surfactant, a sugar, a polymer, and combination thereof.

In another preferred embodiment, the pharmaceutical composition may comprise other active ingredients in addition to the peptides of the invention. In one embodiment, the other active ingredient may be an antiviral or antibacterial agent, for the treatment or prevention of secondary infection in the subject. Antiviral drugs include, but not limited to, oseltamivir (Tamiflu), zanamivir (Relenza®), permivir (Rapivab®), dideoxynucleosides, azidothymadine, Ribavirin, Interferon and the like.

As used herein, a “pharmaceutically acceptable carrier” refers to a carrier or diluent that does not cause significant irritation to an organism, does not abrogate the biological activity and properties of the administered active ingredient, and/or does not interact in a deleterious manner with the other components of the composition in which it contains. The term “carrier” encompasses any excipient, binder, diluent, filler, salt, buffer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g. Remington's Pharmaceutical Sciences, 21st Edition, ed. University of the Sciences in Philadelphia, Lippincott, Williams & Wilkins, Philadelphia Pa., 2005, which is incorporated herein by reference in its entirety. Examples of physiologically acceptable carriers include antioxidants including ascorbic acid; low molecular weight (less than about 10 residues) peptides; proteins, such as serum albumin, gelatine, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrin; chelating agents such as EDTA; sugar alcohols such as mannitol or sorbitol; salt-forming counter ions such as sodium; and/or non-ionic surfactants such as TWEEN° (ICI, Inc.; Bridgewater, N.J.), polyethylene glycol (PEG), and PLURONICS™ (BASF; Florham Park, N.J.). An “excipient” refers to an inert substance added to a composition to further facilitate administration of a compound. Examples, without limitation, of excipients include calcium carbonate, calcium phosphate, various sugars and types of starch, cellulose derivatives, gelatine, vegetable oils, and polyethylene glycols.

In some embodiments, the pharmaceutically acceptable carrier and/or excipient is at least one selected from the group consisting of a buffer, an inorganic salt, a fatty acid, a vegetable oil, a synthetic fatty ester, a surfactant, and a polymer.

Exemplary buffers include, without limitation, phosphate buffers, citrate buffer, acetate buffers, borate buffers, carbonate/bicarbonate buffers, and buffers with other organic acids and salts.

Exemplary inorganic salts include, without limitation, calcium carbonate, calcium phosphate, disodium hydrogen phosphate, potassium hydrogen phosphate, sodium chloride, zinc oxide, zinc sulfate, and magnesium trisilicate.

Exemplary fatty acids include, without limitation, an omega-3 fatty acid (e.g., linolenic acid, docosahexaenoic acid, eicosapentaenoic acid) and an omega-6 fatty acid (e.g., linoleic acid, eicosadienoic acid, arachidonic acid). Other fatty acids, such as oleic acid, palmitoleic acid, palmitic acid, stearic acid, and myristic acid, may be included.

Exemplary vegetable oils include, without limitation, avocado oil, olive oil, palm oil, coconut oil, rapeseed oil, soybean oil, corn oil, sunflower oil, cottonseed oil, and peanut oil, grape seed oil, hazelnut oil, linseed oil, rice bran oil, safflower oil, sesame oil, brazil nut oil, carapa oil, passion fruit oil, and cocoa butter.

Exemplary synthetic fatty esters include, without limitation, methyl, ethyl, isopropyl and butyl esters of fatty acids (e.g., isopropyl palmitate, glyceryl stearate, ethyl oleate, isopropyl myristate, isopropyl isostearate, diisopropyl sebacate, ethyl stearate, di-n-butyl adipate, dipropylene glycol pelargonate), C₁₂-C₁₆ fatty alcohol lactates (e.g., cetyl lactate and lauryl lactate), propylene dipelargonate, 2-ethylhexyl isononoate, 2-ethylhexyl stearate, isopropyl lanolate, 2-ethylhexyl salicylate, cetyl myristate, oleyl myristate, oleyl stearate, oleyl oleate, hexyl laurate, isohexyl laurate, propylene glycol fatty ester, and polyoxyethylene sorbitan fatty ester. As used herein, the term “propylene glycol fatty ester” refers to a monoether or diester, or mixtures thereof, formed between propylene glycol or polypropylene glycol and a fatty acid. The term “polyoxyethylene sorbitan fatty ester” denotes oleate esters of sorbitol and its anhydrides, typically copolymerized with ethylene oxide.

Surfactants may act as detergents, wetting agents, emulsifiers, foaming agents, and dispersants. Surfactants that may be present in the compositions of the present disclosure include zwitterionic (amphoteric) surfactants, e.g., phosphatidylcholine, and 3-[(3-cholamidopropyl)dimethylammonio]-1-propanesulfonate (CHAPS), anionic surfactants, e.g., sodium lauryl sulfate, sodium octane sulfonate, sodium decane sulfonate, and sodium dodecane sulfonate, non-ionic surfactants, e.g., sorbitan monolaurate, sorbitan monopalmitate, sorbitan trioleate, polysorbates such as polysorbate 20 (Tween 20), polysorbate 60 (Tween 60), and polysorbate 80 (Tween 80), cationic surfactants, e.g., decyltrimethylammonium bromide, dodecyltrimethylammonium bromide, tetradecyltrimethylammonium bromide, tetradecyltrimethylammonium chloride, and dodecylammonium chloride, and combinations thereof.

Exemplary polymers include, without limitation, polylactides, polyglycolides, polycaprolactones, polyanhydrides, polyurethanes, polyesteramides, polyorthoesters, polydioxanones, polyacetals, polyketals, polycarbonates, polyorthocarbonates, polyphos-phazenes, polyhydroxybutyrates, polyhydroxyvalerates, polyalkylene oxalates, polyalkylene succinates, poly(malic acid), poly(maleic anhydride), a polyvinyl alcohols, and copolymers, terpolymers, or combinations or mixtures therein. The copolymer/terpolymer may be a random copolymer/terpolymer, or a block copolymer/terpolymer.

Depending on the route of administration e.g. oral, parental, or topical, the composition may be in the form of solid dosage form such as tablets, caplets, capsules, powders, and granules, semi-solid dosage form such as ointments, creams, lotions, gels, pastes, and suppositories, liquid dosage forms such as solutions, and dispersions, inhalation dosage form such as aerosols, and spray, or transdermal dosage form such as patches.

Solid dosage forms for oral administration can include capsules, tablets, pills, powders, and granules. In such solid dosage forms, the active ingredient is ordinarily combined with one or more adjuvants appropriate to the indicated route of administration. If administered per os, the active ingredient can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatine, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering ingredients such as sodium citrate, magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting ingredients, emulsifying and suspending ingredients, and sweetening, flavouring, and perfuming ingredients.

For therapeutic purposes, formulations for parenteral administration can be in the form of aqueous or non-aqueous isotonic sterile injection solutions or suspensions. The term “parenteral”, as used herein, includes intravenous, intravesical, intraperitoneal, subcutaneous, intramuscular, intralesional, intracranial, intrapulmonal, intracardial, intrasternal, and sublingual injections, or infusion techniques. These solutions and suspensions can be prepared from sterile powders or granules having one or more of the carriers or diluents mentioned for use in the formulations for oral administration. The active ingredient can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Injectable preparations, for example, sterile injectable aqueous or oleaginous suspensions can be formulated according to the known art using suitable dispersing or wetting ingredients and suspending ingredients. The sterile injectable preparation can also be a sterile injectable solution or suspension in a non-toxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids, such as oleic acid, find use in the preparation of injectable. Dimethylacetamide, surfactants including ionic and non-ionic detergents, polyethylene glycols can be used. Mixtures of solvents and wetting ingredients such as those discussed above are also useful.

Suppositories for rectal administration can be prepared by mixing the active ingredient with a suitable non-irritating excipient, such as cocoa butter, synthetic mono-, di-, or triglycerides, fatty acids, and polyethylene glycols that are solid at ordinary temperatures but liquid at the rectal temperature and will therefore melt in the rectum and release the drug.

Topical administration may involve the use of transdermal administration such as transdermal patches or iontophoresis devices. Formulation of drugs is discussed in, for example, Hoover, J. E. Remington's pharmaceutical sciences, Mack Publishing Co., Easton, PA, 1975; and Liberman, H. A.; Lachman, L., Eds. Pharmaceutical dosage forms, Marcel Decker, New York, N.Y., 1980, which are incorporated herein by reference in their entirety.

In other embodiments, the composition comprising the antiviral peptides disclosed herein has different release rates categorized as immediate release and controlled- or sustained-release.

As used herein, immediate release refers to the release of an active ingredient substantially immediately upon administration. In another embodiment, immediate release occurs when there is dissolution of an active ingredient within 1-20 minutes after administration. Dissolution can be of all or less than all (e.g. about 70%, about 75%, about 80%, about 85%, about 90%, about 91%, about 92%, about 93%, about 94%, about 95%, about 96%, about 97%, about 98%, about 99%, about 99.5%, 99.9%, or 99.99%) of the active ingredient. In another embodiment, immediate release results in complete or less than complete dissolution within about 1 hour following administration. Dissolution can be in a subject's stomach and/or intestine. In one embodiment, immediate release results in dissolution of an active ingredient within 1-20 minutes after entering the stomach. For example, dissolution of 100% of an active ingredient can occur in the prescribed time. In another embodiment, immediate release results in complete or less than complete dissolution within about one hour following rectal administration. In some embodiments, immediate release is through inhalation, such that dissolution occurs in a subject's lungs.

Controlled-release, or sustained-release, refers to a release of an active ingredient from a composition or dosage form in which the active ingredient is released over an extended period of time. In one embodiment, controlled-release results in dissolution of an active ingredient within 20-180 minutes after entering the stomach. In another embodiment, controlled-release occurs when there is dissolution of an active ingredient within 20-180 minutes after being swallowed. In another embodiment, controlled-release occurs when there is dissolution of an active ingredient within 20-180 minutes after entering the intestine. In another embodiment, controlled-release results in substantially complete dissolution after at least 1 hour following administration. In another embodiment, controlled-release results in substantially complete dissolution after at least one hour following oral administration. In another embodiment, controlled-release results in substantially complete dissolution after at least one hour following rectal administration. In one embodiment, the composition is not a controlled-release composition.

Methods of Treatment and Protection from MERS-CoV Infection

A third aspect of the invention is directed to a method of treatment of a subject infected with HCV, or protecting a subject from getting infected by HCV. The method comprises administering to a subject infected with HCV an effective amount of the pharmaceutical composition described here. Treatment is preferably commenced at the time of infection or post infection with HCV. It is recommended that the treatment continues until the virus is no longer present or active. For protecting a non-infected subject from future infection, the treatment continues for as long as there is a potential exposure to the virus.

As used herein the term “effective amount” refers to an amount of a pharmaceutical composition administered to a subject that is sufficient to provide relief from the symptoms of HCV infection. The effective amount of the pharmaceutical composition administered to a subject varies and is dependent on the age and weight of the subject as well as the severity of the infection. Suitable treatment is given 1-4 times daily and continued for 3-10 days, and typically 8 days post infection. The desired dose may be presented in a single dose or as divided doses administered at appropriate intervals, for example as two, three, four or more sub-doses per day. The pharmaceutical composition may be conveniently administered in unit dosage form, wherein the peptide content of the pharmaceutical composition is in the range of 10 to 1500 mg, conveniently 20 to 1000 mg, most conveniently 50 to 700 mg of active ingredient per unit dosage. Usually, the dose of the peptide is in the range of 1 mg/kg to 150 mg/kg of body weight, preferably in the range of 25 mg/kg to 100 mg/kg of body weight, more preferably in the range of 50 mg/kg to 90 mg/kg of body weight, and most preferably in the range of 70 mg/kg to 80 mg/kg of body weight.

### Method of Identifying Antiviral Compounds

A fourth aspect of the invention is directed a method of designing, identifying, selecting, and optimizing an antiviral peptide selected from the group consisting of SEQ ID NO: SEQ ID NO: 4, 9, 10, 14, 15, 19, and 20 to obtain a chemical compound having increased antiviral activity relative to the parent peptide. The method comprises:

(a) construct a three dimensional model of the HCV NS3 protease's activation site in silico using a set of atomic coordinates of Protein Data Bank accession number selected from the group consisting of 1NS3, 2OIN, 2OBO, 2O8M, 2OBQ, and 2OC14KQZ—incorporated herein by reference in their entirety,

(b) dock a peptide selected from SEQ ID NO: SEQ ID NO: 4, 9, 10, 14, 15, 19, and 20 to the binding site of the model,

(c) modify the structure of the peptide sequence to enhance and optimize the interactions between the peptide and the receptor binding domain of spike protein,

(d) synthesize the resulting peptide or compound,

(e) measure the binding of the peptide or compound to an HCV NS3 protease of SEQ ID NO: 1 or a variant thereof having at least 60%.

The structure HCV NS3 protease with and without the activation peptide is fully disclosed and describe, see for example Prongay et al. [J. Med. Chem (2007) 50, 2310-2318], Zhou et al. [J. Biol. Chem (2007) 282, 22619-22628], and Bogen et al. [Bioorg. Med. Che, (2006) 16, 1621-1627]—incorporated herein by reference in their entirety. The method of the invention structure guided method for modifying one of the peptides of SEQ ID NO: 4, 9, 10, 14, 15, 19, and 20 to produce a chemical compound or a peptide with improved binding characteristics and pharmacokinetic properties. As used herein, the words “design” or “designing” is meant to provide a novel molecular structure of, for example, a compound, such as a small molecule or a substrate analogue of the peptides of SEQ ID NO: 4, 9, 10, 14, 15, 19, and 20. The resulting molecule may be any chemical entity that binds to the activation site of HCV NS3 protease such as but not limited to linear peptides, cyclic peptides, macrolactons, macrolactams, and peptidomimetics. Suitable computer programs which may be used in the design and identification of potential binding compounds (e.g., by selecting suitable chemical fragments) include, but are not limited to, GRID [Goodford 1985 J. Med. Chem. 28:849 857], MCSS [Miranker, A. and M. Karplus, (1991) Proteins: Structure. Function and Genetics, 11:29-34], AUTODOCK [Goodsell, D. S et al (1990) Proteins: Structure. Function, and Genetics 8:195 202]; and DOCK [Kuntz, I. D. et al. (1982) J Mol. Biol 161:269-288; and Bartlett, (1989) Molecular Recognition in Chemical and Biological Problems, Special Pub., Royal Chem. Soc. 78:182-196]. Suitable computer programs which may be used in connecting the individual chemical entities or fragments include, but are not limited to, CAVEAT (Bartlett, (1989) Molecular Recognition in Chemical and Biological Problems, Special Pub., Royal Chem. Soc. 78:182-19632); and 3D Database systems such as MACCS-3D by MDL Information Systems, San Leandro, Calif), HOOK (Molecular Simulations, Burlington, Mass.) and as reviewed in reference [Martin, Y. C, (1992) J Med. Chem, 35:2145 2154]. Other suitable computer programs which may be used to modify the peptides of the invention include, but not limited to, LUDI [Bohrn, (1992) J. Comp. Aid Molec. Design 6:61-78], LEGEND [Nishibata et al. (1991) Tetrahedron 47:8985]; and LEAPFROG [Tripos Associates, St. Louis, Mo.]. Also, other molecular modeling techniques may be employed in accordance with this invention [Cohen, N. C. et al. (1990) J Med. Chem. 33: 883-894 incorporated herein by reference in its entirety; and Navia (1992) Current Opinions in Structural Biology 2:202-210 incorporated herein by reference in its entirety]. A potential binding compound has been designed, selected, identified, synthesized, or chosen by the methods described herein, the affinity with which that a compound binds to the activation site may be tested and optimized by computational evaluation. A compound designed, or selected, or synthesized, or chosen as potential binding compound or may be further computationally optimized so that in its bound state it would preferably lack repulsive electrostatic interaction with the target activation site. Such non-complementary (e.g., electrostatic) interactions include repulsive charge-charge, dipole-dipole and charge-dipole interactions. Specifically, the sum of all electrostatic interactions between the potential binding compound and the binding site is neutral or make favorable contribution to the enthalpy of binding. Suitable computer software which may be used to evaluate compound deformation energy and electrostatic interactions, includes, but is not limited to, Gaussian 92, revision C [M. J. Frisch, Gaussian, Inc., (1992) Pittsburgh, Pa.]; AMBER, version 4.0 [P. A. Kollman, (1994) University of California at San Francisco]; QUANTA/CHARMM [Molecular Simulations, Inc., (1994) Burlington, Mass].; and Insight II/Discover [Biosysm Technologies Inc., (1994) San Diego, Calif.]. These programs may be implemented, for example, using a Silicon Graphics workstation, IRIS 4D/35 or IBM RISC/6000 workstation model 550. Hardware systems, such as an IBM thinkpad with LINUX operating system or DELL latitude D630 with WINDOWS operating system, may be used. Other hardware systems and software packages will be known to those skilled in the art of which the speed and capacity are continually modified.

As used herein, a “binding compound” refers to a compound which reversibly or irreversibly binds to HCV NS3 protease or variant thereof having amino acid sequence at least 60% sequence identity to SEQ ID NO: 1 at the activation site. Binding may involve the formation of bonds which may be covalent or non-covalent. Non-covalent bonds may be e.g. hydrogen bonds, ionic bonds or hydrophobic interactions. A binding compound is expected to interfere and inhibit the interactions leading to the formation of the active form of the NS3 protease.

A binding compound may be a small molecule. The term “small molecule” as used herein is meant to describe a low molecular weight organic compound which is not a polymer. A small molecule may bind with high or low affinity to a biopolymer such as protein, nucleic acid, or polysaccharide and may in addition alter the activity or function of the biopolymer. The molecular weight of the small organic compound may generally be smaller than about 2500 Da. Small molecules may be smaller than about 2000 Da, smaller than about 1000 Da, or smaller than about 800 Da. Small molecules may rapidly diffuse across cell membranes and may have oral bioavailability.

It is useful to be able to identify binding molecules that are specific to the activation site of HCV NS3 protease of SEQ ID NO: 1 or variants thereof having at least 60% sequence identity to SEQ ID NO: 1. By specific, it is meant that the binding molecule has a preference for binding to the activation site of HCV NS3 protease of SEQ ID NO: 1 or variants thereof having at least 60% sequence identity to SEQ ID NO: 1, and does not bind to one or more other biomolecules or shows at least 5, 10, 20, 50, 100, 200, 500, or 1000 fold reduced affinity to one or more other biomolecules. Binding can be quantitated in accordance with methods well-known in the art and described herein below. Furthermore, in certain embodiments, the above method further comprises the steps of using a suitable assay, as described herein, to characterize the potential binding compound's ability to bind to the activation site. This may involve directly testing the compound's ability to bind, and/or determining whether the compound has an influence on the binding of the NS4 to HCV NS3 protease of SEQ ID NO: 1 or variants thereof having at least 60% sequence identity to SEQ ID NO: 1. To evaluate binding properties of binding compounds, assays may be used. Several assay methods are well-known in the art. The methods include, but not limited to, calorimetric techniques, surface plasmon resonance (SPR, Biacore™), kinetics methods, and spectroscopic methods including NMR methods, fluorescence methods and UV-Vis methods.

Calorimetric methods include but not limited to isothermal titration calorimetry and differential scanning calorimetry. SPR is the resonant oscillation of conduction electrons at the interface between negative and positive permittivity material stimulated by incident light. The method involves immobilizing one molecule of a binding pair on the sensor chip surface (“ligand”, in Biacore parlance) and injecting a series of concentrations of its partner (“analyte”) across the surface. Changes in the index of refraction at the surface where the binding interaction occurs are detected by the hardware and recorded as RU (resonance units) in the control software. Curves are generated from the RU trace and are evaluated by fitting algorithms which compare the raw data to well-defined binding models. These fits allow determination of a variety of thermodynamic constants, including the apparent affinity of the binding interaction. SPR main advantage is that it does not require labeling the protein or the binding compound.

The kinetics of enzymatic-catalyzed reactions is a useful tool not only to determine the inhibition constants (Ki) for an inhibitor but also the site at which an inhibitor binds to the enzyme. Any standard text book in enzymology describes in details the methodology, see for example Fersht, A. [“Enzyme Structure and Mechanism” (1985) chapter 3, pp 98-120, W. H. Freeman, New York] and Creighton, T. E. [“Proteins Structures and Molecular Properties” (1993) second Edition, Chapter 9, pages 385-392]—both incorporated herein their entirety. An inhibitor that binds exclusively to the catalytic active site displays a competitive inhibition pattern with the substrate. In contrast, an inhibitor that binds to a different site from that of the substrate displays an uncompetitive inhibition pattern with the substrate. If the inhibitor binds to both an active site and a different site from that of the active site, it would display a non-competitive pattern. Thus, the inhibitor of the invention would be competitive with the activation peptide and uncompetitive with the substrate. Since the substrates of the enzyme are peptides, some peptides of the invention may bind to both the activation site and the catalytic active site of an HCV NS3 protease having at least 60% sequence identity to SEQ ID NO: 1 and in such a case a non-competitive kinetic pattern should be observed.

NMR methods and optical spectroscopic methods such as fluorescence, UV-Vis, and Circular Dichroism are well-known method utilized in measuring the interaction between a binding compound and a protein. The fluorescence method is suitable for high throughput screening method amenable to automation in a laboratory environment. Since HCV NS3 of SEQ ID NO: 1 contains two tryptophan residues and four tyrosine residues, the binding of a peptide inhibitor to the activation site may be accompanied by significant change in the intrinsic fluorescence of the protein, and hence the binding constant may be obtained. Any peptide inhibitor may be labeled with a fluorescent probe and the binding of the labeled peptide to the enzyme is accompanied by fluorescent change, see examples below. Another fluorescence assay method for determining the binding constant of the peptide inhibitors of the invention is a competitive displacement assay method describe herein in the examples.

NMR methods may be use to observe directly the binding of a peptide inhibitor of activation to the activation site of HCV NS3 protease of SEQ ID NO: 1 and valuable structural information may be obtained in addition to the binding constant. In its simple form, the observation of broadening of an NMR signal as a function of concentration would allow the determination of binding constants. Some other NMR methods may require isotopically labeled binding compounds and/or proteins. Methods of obtaining isotopically labelled proteins and binding compounds with ²H, ¹³C, and ¹⁵N are well-known in the art. ²H, ¹³C, and ¹⁵N proteinogenic amino are commercially available and can be incorporated in a culture medium to obtain labeled enzyme. Also, labeled protected amino acids suitable for peptide synthesis are commercially available.

### EXAMPLE 1

### Material and Method

Unless otherwise indicated, all chemicals were molecular biology grade and purchased from Sigma Aldrich (St Louis, Mo., USA).

Synthetic peptide comprising the activation sequence residue 21 to 33 of SEQ ID NO: 2 of NC4A wild-type of SEQ ID NO: 4 and fluorescein isothiocyanate-NS4A (FITC-NS4A), as well as variants thereof SEQ ID NO: 6-20 were obtained from GenScript (Hong Kong). Also, the synthetic variants of SEQ ID NO: 4 were custom synthesized by Bio-Synthesis Inc. (Lewisville, Tex., US) shown below in Table 1. All synthetic peptides were 85% purity or more (LC/MS).

TABLE 1

| | | Hepatitis | | |
|---|---|---|---|---|
| NS4A | SEQ ID NO: 4 | C virus | KKGSVVIVGRIVLSGKK | |
| Pep-X | SEQ ID NO: 5 | | GSX₁VX₂VGRX₃VLSG | |
| Pep-1 | SEQ ID NO: 6 | synthetic | KKGSVVIVGRFVLSGKK | Ile-29 variants |
| Pep-2 | SEQ ID NO: 7 | synthetic | KKGSVVIVGRWVLSGKK | |
| Pep-3 | SEQ ID NO: 8 | synthetic | KKGSVVIVGRAVLSGKK | |
| Pep-4 | SEQ ID NO: 9 | synthetic | KKGSVVIVGRhIVLSGKK⁽¹⁾ | |
| Pep-5 | SEQ ID NO: 10 | synthetic | KKGSVVIVGRxGVLSGKK⁽²⁾ | |
| Pep-6 | SEQ ID NO: 11 | synthetic | KKGSVVFVGRIVLSGKK | |
| Pep-7 | SEQ ID NO: 12 | synthetic | KKGSVVWVGRIVLSGKK | Ile-25 variants |
| Pep-8 | SEQ ID NO: 13 | synthetic | KKGSVVAVGRIVLSGKK | |
| Pep-9 | SEQ ID NO: 14 | synthetic | KKGSVVhIVGRIVLSGKK⁽¹⁾ | |
| Pep-10 | SEQ ID NO: 15 | synthetic | KKGSVVxGVGRIVLSGKK⁽²⁾ | |
| Pep-11 | SEQ ID NO: 16 | synthetic | KKGSFVIVGRIVLSGKK | Val-23 variants |
| Pep-12 | SEQ ID NO: 17 | synthetic | KKGSWVIVGRIVLSGKK | |
| Pep-13 | SEQ ID NO: 18 | synthetic | KKGSAVIVGRIVLSGKK | |
| Pep-14 | SEQ ID NO: 19 | synthetic | KKGShIVIVGRIVLSGKK⁽¹⁾ | |
| Pep-15 | SEQ ID NO: 20 | synthetic | KKGSxGVIVGRIVLSGKK⁽²⁾ | |

⁽¹⁾Residue hI is homoisoleucine [(2S)-2-amino-4-methylhexanoic acid]
⁽²⁾Residue xG is (S)-cyclohexylglycine.

A synthetic gene coding for the HCV NS3 domain of genotype 4a SEQ ID NO: 1, the most abundant HCV in Saudi Arabia and Egypt, was synthesized by GenScript (Hong Kong).

The wild-type nucleic acid sequence was codon optimized for expression in E. coli [Massariol et al. (2010) “Protease and helicase activities of hepatitis C virus genotype 4, 5, and 6 NS3-NS4A proteins” Biochemical and Biophysical Research Communications, 391, 692-697, incorporated herein by reference in its entirety]. The synthetic gene was inserted into the cloning site NdeI-BamHI of expression vector pET-3a Novagen® and the vector was sequenced to confirm its structure.

### EXAMPLE 2

### Protein Expression

The fusion protein of SEQ ID NO: 3 consisting of SEQ ID NO: 1 of the NS3 domain of Hepatitis C virus (genotype 4a) fused to the T7 tag at the N-terminus and 6-His tag at the C-terminus was expressed in E. coli Rosette (DE3) pLysS as described by Kim et al. (1996)-incorporated herein by reference in its entirety. A synthetic nucleic acid sequence encoding the NS3 domain was subcloned into the expression vector pET-3a. A bacterial culture in 100 mL Luria Broth medium grew overnight at 37° C. and used for inoculation of 10 L LB in a 14-liter fermenter flask (New Brunswick Scientific Co., Conn., USA). The media was supplemented with ampicillin 50 μg/mL. The culture grew until the OD₆₀₀ reached 0.5-0.6, then it was cooled to 25° C. and 1mM IPTG was added. The expression continued at 37 ° C., overnight and then cells were harvested.

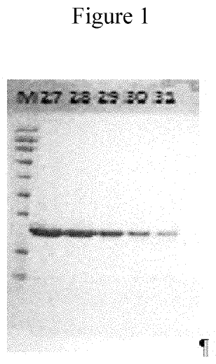

Cells (1.0 g) were suspended in 5 mL 50 mM HEPES containing 0.3M NaCl, 10% glycerol, and 2 mM β-mercaptoethanol at pH8. Lysozyme was added to a concentration of 1 mg/mL, followed by protease inhibitor cocktail tablet and the suspension was sonicated. Cell lysate was centrifuged and the supernatant containing the expressed protein was collected. The supernatant was loaded on a column packed with Ni-NTA beads (Qiagen, USA) and equilibrated with 50 mM HEPES containing 0.3 M NaCl, 10% glycerol, 2 mM β-mercaptoethanol, and 20 mM imidazole at pH8 buffer. The column was eluted 50 mM HEPES buffer containing 0.3M NaCl, 10% glycerol, 2 mM (3-mercaptoethanol, and 350 mM imidazole at pH8. Fractions were collected and concentrated using Amicon Ultra-4 3000 MWCO centrifugal filtering unit (Millipore, Germany). The purity of the protein of SEQ ID NO: 3 eluted from Ni-NTA column was determined to be at least 70% by SDS-PAGE (see FIG. 1), use without further purification in most experiments (Massariol et al. (2010)-incorporated herein by reference in its entirety. The final concentration of SEQ ID NO: 3 was determined spectrophotometrically at 280 nm using Nanodrop™ nanoscale spectrophotometer.

Samples of described above enzyme were further purified on a Superdex 75 (16/90 column, GE Healthcare, USA) eluted with 20 mM HEPES, 10 mM DDT, 200 mM NaCl at pH 7.6 at a flow rate of 1 mL/min and purity was estimated using SDS-PAGE.

### EXAMPLE 3

The binding of SEQ ID NO: 4 and its variants of SEQ ID NO: 6-20 to the NS3 of SEQ ID NO: 3 were determined by differential static light scattering method using Stargazer-2™ (Harbinger Biotechnology and Engineering Corporation, Toronto, Canada). The method assesses protein stability by monitoring aggregate formation upon gradual increase of temperatures. NS3 domain of SEQ ID NO: 3 stability upon binding to NS4A of SEQ ID NO: 4 was measured by monitoring denatured protein aggregation upon increasing temperature from 25 to 85° C. in 0.5° C. increments at 600 nm. In a typical measurement, 10 μL of 150 μM NS3 of SEQ ID NO: 3 was added to 0.08 mL of the binding buffer containing 20 mM HEPES, 10 mM DTT, and 200 mM, NaCl at pH 7.6 and followed by the addition of 10 μL of 150 μM solution of a synthetic peptide. In a control measurement, 10 μL of 150 μM NS3 of SEQ ID NO: 3 was added to 0.09 mL of the binding buffer containing 20 mM HEPES, 10 mM DTT, and 200 mM, NaCl at pH 7.6. The mixture and control were incubated at room temperature with gentle shaking for specified time. For control measurement. Afterwards, 10 μL of the mixture was transferred into a clear bottom Nunc 384-well plate and covered by 10 μL paraffin oil to minimize evaporation. Protein aggregation was monitored by tracking the change in scattered light that was detected by a CCD camera. Snapshot images of the plate were taken every 0.5° C. The pixel intensities in a preselected region of each well were integrated using image analysis software to generate a value representative of the total amount of scattered light in that region. The intensities were then plotted against temperature for each sample well and fitted to obtain the aggregation temperature (T_{agg}). Aggregation was monitored and analyzed to assess the effect of NS4A of SEQ ID NO: 4 and its synthetic analogues of SEQ ID NO: 6-20 on the stability of the NS3 as an indicator of binding.

Fluorescence anisotropy was used to quantify dissociation constant (K_{d}) of NS4A of SEQ ID NO: 25 and Pep-15 of SEQ ID NO: 20. In 96-well plate, serial dilutions of NS3 of SEQ ID NO: 3 were prepared in binding buffer containing 20 mM HEPES, 10 mM DTT, and 200 mM NaCl at pH 7.6. To the enzyme solutions, 0.1 μM isothiocyanate-labeled NS4A peptide of SEQ ID NO: 25 (FITC-NS4A) was added and agitated for 15, 45, 90 and 120 min. at room temperature. A volume of 20 μL of the solution comprising the NS3 protein of SEQ ID NO: 3 and FITC-NS4A SEQ ID NO: 25 was transferred to a black reading Nunc 384-well plate. Fluorescence was observed at 520 nm using excitation wavelength of 480 nm of PHERAstar™ plate reader (BMG Labtech, Ortenberg, Germany). Emitted fluorescence was proportional to the concentration of FITC-NS4A/NS3 complex (bound form). A competition assay was used to measure the binding assay of Pep-15 of SEQ ID NO: 20. In a typical measurement, varying concentration of Pep-15 of SEQ ID NO: 20 in the range of 100 μM-0.195 μM were added to solutions containing 1.8 μM NS3 of SEQ ID NO 21 and 0.1 μM FITC-NS4A, and the fluorescence of the solutions were observed at 520 nm using excitation wavelength of 480 nm. The synthetic variant of SEQ ID NO: 20 displayed higher affinity for the protein than that of the native NS4A of labeled SEQ ID NO: 4.

The dissociation constant (K_{d}) was calculated using the non-linear regression equation in GraphPad Prism version 7.00 for Windows, GraphPad Software, La Jolla Calif. USA.

### EXAMPL.E 4

### Protease Assay

The protease assay was performed using SensoLyte520® HCV protease assay kit fluorometric (Anaspec, Fremont, Calif., USA) according to a modified procedure to suite the purpose of determination of allosteric inhibition. NS3 of SEQ ID NO: 3 (4.0 μM) was mixed with variable concentrations from 0.001 to 50 μM of Pep-15 of SEQ ID NO: 20 for 15 minutes. Afterwards, 5-FAM/QXL™520 fluorescence resonance energy transfer (FRET) peptide was added as instructed by the assay kit manual. The sequence of the FRET peptide of SEQ ID NO: 20 is 5-FAM-SLGRKIQIQ-QXL™520 of SEQ ID NO: 22 which is derived from the cleavage site of NS4A/NS4B. In the FRET peptide of SEQ ID NO: 22, the fluorescence of 5-FAM is quenched by QXL™520. Upon cleavage of the peptide, the two chromophores are separated, and the fluorescence of 5-FAM is revealed, which can be monitored at 520 nm using excitation wave length of 480 nm.

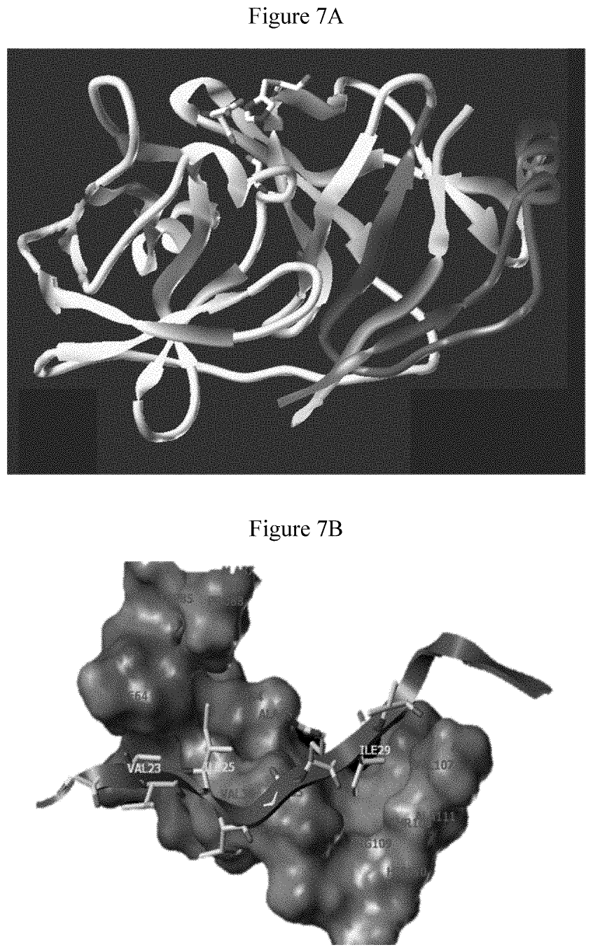

Crystal structure studies show that the activation peptide NS4A of SEQ ID NO: 2 is bound to NS3 of SEQ ID NO: 1 is in an extended conformation except for the backbone bond between the α-carbon and the carbonyl group of the peptide bond of Val-26 of SEQ ID NO: 2 (see FIGS. 7A and 7B).

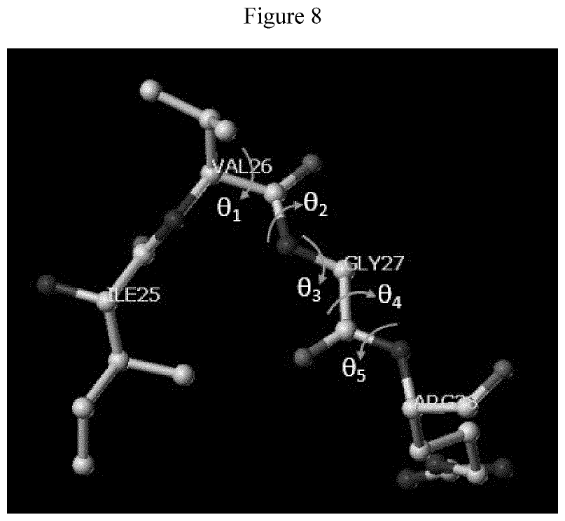

The turn is conserved in all structures of NS3/NS4A found in the Protein Databank (PDB) [Love et al. (1996) “The crystal structure of hepatitis C virus NS3 proteinase reveals a trypsin-like fold and a structural zinc binding site” Cell, 87, 331-342; Hagel et al. (2011) “Selective irreversible inhibition of a protease by targeting a noncatalytic cysteine” Nature chemical biology, 7, 22; Kim et al. (1996); and Prongay et al. (2007) “Discovery of the HCV NS3/4A Protease Inhibitor (1R,5S)-N[3-amino-1-(cyclobutylmethyl)-2,3-dioxopropyl]-3-[2(S)-[[[(1, 1-dimethylethyl)amino]-carbonyl]amino]-3 ,3 -dimethyl-1-oxobutyl]-6,6-dimethyl-3-azabicyclo[3.1.0]hexan-2(S)-carboxamide (Sch 503034) II. Key Steps in Structure-Based Optimization” Journal of Medicinal Chemistry, 50, 2310-2318-each incorporated herein by reference in their entirety. For example, the PDB accession number 1NS3 and Yan et al. [Protein Sci. (1998) 7, 837-847--incorporated herein by reference in its entirety] shows the dihedral angle ψ of Val 26 is 14° (shown as θ₁ in FIG. 8) leading to a confirmation where the side chain of Val-26 eclipses the two closest imido hydrogens. The other three dihedral angles θ₂, θ₃, and θ₄ in FIG. 8 are anti-conformation of β-sheet approaching 180° (see FIG. 8). Table 2 below summarizes the dihedral angles shown in FIG. 8.

TABLE 2
Dihedral angles (Torsion) of core part of NS4A

| Torsion | Actual | Deviation from Plane |
|---|---|---|
| θ₁ (ψ₁) | 13.9 | +13.9 |
| θ₂ (Φ₁) | 179.4 | −0.6 |
| θ₃ (ψ₂) | 184.6 | +4.6 |
| θ₄ (Φ₂) | 191.6 | +11.6 |
| θ₅ (ψ₃) | 184.3 | +4.3 |

In the context, this may explain the required presence of Gly-27 of SEQ ID NO: 2 in the sequence for binding and activation of NS3. Therefore, peptides variants at a position corresponding Gly-27 of SEQ ID NO: 2 were excluded from the study.

The three-dimensional structure of NS3/NS4a shows that the even numbered residues of NS4A of SEQ ID NO: 2 (Ser-22, Val-24, Val-26, Arg-28, Val-30 and Ser-32) are interacting with the A₀ β-sheet at the N-terminus of the NS3 protein of SEQ ID NO: 1 and exposed to the solvent. In contrast, the NS4A of SEQ ID NO: 2 odd-numbed residues (Val-23, Ile-25, Gly-27, Ile-29 and Leu-31) are interacting with the A₁ β-sheet (residues Glu-58 to Ser-63 of SEQ ID NO: 1), and are mostly buried within the protein core [Yan et al. (1998)-incorporated herein by reference in its entirety]. Thus, a working hypothesis was formulated that bulkier hydrophobic variants of residues Val-23, Ile-25 and Ile-29 of SEQ ID NO: 2 may be capable of perturbing the conformation of NS3 binding pockets of SEQ ID NO: 1 of the activation peptide of SEQ ID NO: 2, and thereby preventing the enzyme from assuming the active conformation geometry required for catalytic activity. As indicated earlier, the peptide of SEQ ID NO: 4 which comprises residues 21 to 33 of NS4a of SEQ ID NO: and is sufficient to activate the NS3 protease of SEQ ID NO: 1, 15 peptide variants of SEQ ID NO: 4 were tested for their inhibition of NS3 protease of SEQ ID NO: 3. The variant peptides contain an amino acid substitution with bulkier side chains (see Table 1). The non-proteinogenic amino acids (S)-cyclohexylglycine (xG) and (S)-homoisoleucine (hI) as well as the proteinogenic amino acid Phe and Trp were selected as substituents for Ile and Val because of their bulkier side chain. In addition, the peptide variants contain two lysine residues at the N- and C-termini.

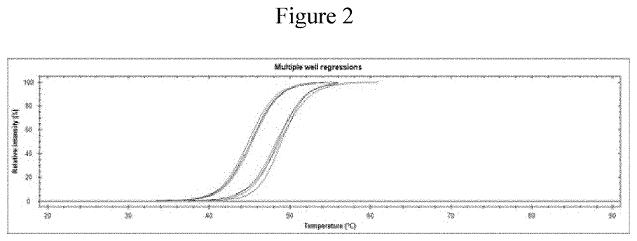

Differential Static Light Scattering (DSLS) was sought because it is a label-free method and would provide preliminary indication about the interaction of the synthetic peptides with the enzyme [Hameed et al. (2018) “Structural basis for specific inhibition of the highly sensitive ShHTL7 receptor” EMBO reports, e45619; and Senisterra et al. (2006) “Screening for ligands using a generic and high-throughput light-scattering-based assay” Journal of biomolecular screening, 11, 940-948, both incorporated herein by reference in their entirety]. DSLS evaluates the non-covalent binding of a ligand to a protein through measuring the protein thermal stability, expressed as shifts in aggregation temperature (T_{agg}) in the absence and the presence of ligand. The highest T_{agg} shift values that are reproducible within acceptable standard errors were obtained by shaking NS3 of SEQ ID NO: 3 with SEQ ID NO: 4 containing the required NS4A sequence for binding and activating the enzyme in 1:2 ratio, respectively, at room temperature for two hours. Under these conditions, NS3/4A binding resulted in a ΔT_{agg} of 2.83±0.12° C., (FIG. 2).

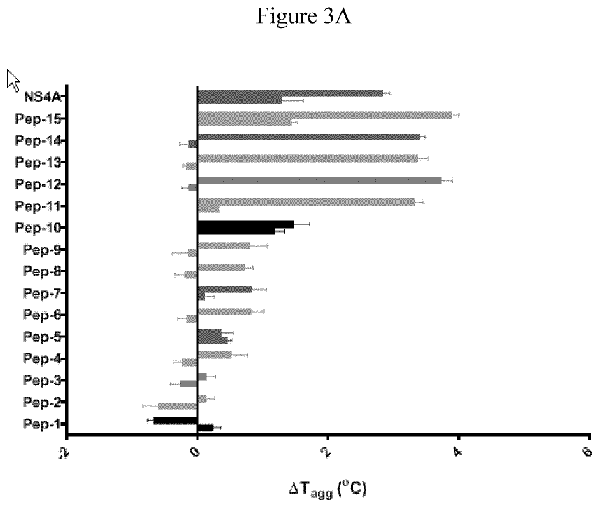

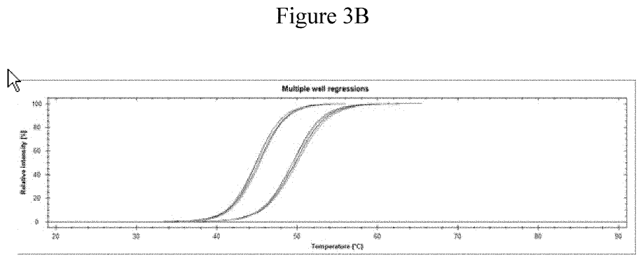

DSLS tests of the synthetic peptides variants performed simultaneously and side-by-side with the peptide of SEQ ID NO: 4. Results revealed that some of disclosed synthetic peptides in Table 1 bind to NS3 of SEQ ID NO: 3. In general, variants at position corresponding Val-23 of SEQ ID NO: 2 displayed higher affinity towards NS3 of SEQ ID NO: 3 compared to those corresponding to positions 25 and 29 of SEQ ID NO: 2 (see FIG. 3A), and have comparable binding to the peptide comprising the native activation sequence of SEQ ID NO: 4.

Pep-15 of SEQ ID NO: 20 complex with the NS3 of SEQ ID NO: 3 exhibited the highest thermal stability not only among the synthetic peptides but also the peptide of SEQ

ID NO: 4 comprising the native activation sequence (FIG. 3A). The T_{agg} shift of Pep-15 of SEQ ID NO: 20 is 3.90° C. compared to 2.83° C. of the peptide of SEQ ID NO: 4 (FIG. 3B). The Kd value for the binding of the peptide of SEQ ID NO: 4 to NS3 of SEQ ID NO: 3 was determined by fluorescence anisotropy. FITC-labelled peptide of SEQ ID NO: 4 (0.1 μM) was mixed with increasing concentrations of unlabeled NS3 of SEQ ID NO: 3. Equation:

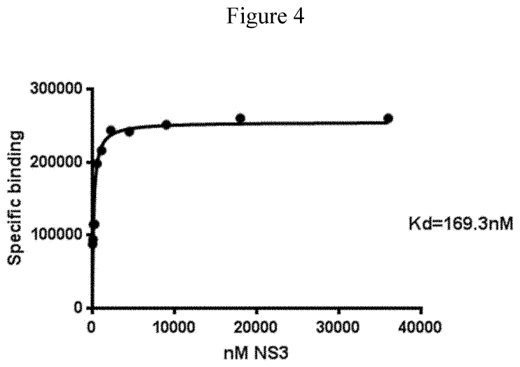

Y=Bₘₐₓ*X/(K_{d}+X)

where Bₘₐₓ is the maximum specific binding in the same units as Y, was fitted to data to produce a K_{d} of 169 ±37 nM (FIG. 4).

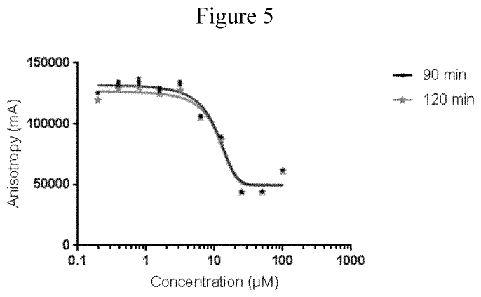

The binding affinity of Pep-15 of SEQ ID NO: 20 to NS3 of SEQ ID NO: 3 was determined using a competition fluorescence anisotropy assay with the fluorescent labeled peptide of SEQ ID NO: 4. The labeled peptide of SEQ ID NO: 4 was mixed with NS3 of SEQ ID NO: 3 at concentrations 0.1 μM and 1.8 μM. Pep-15 of SEQ ID NO: 20 was added at varied concentrations and the fluorescence anisotropy was measured in the present and absence of the peptide. The K_{d} of Pep-15 of SEQ ID NO: 20 was calculated by non-linear fit of a single binding site to data in prism as 70 nM (FIG. 5). It was noted that the complex between Pep-15 of SEQ ID NO: 20 and NS3 of SEQ ID NO: 3 showed excellent stability over significant period of time as indicated by nearly equal fluorescence emissions at 90 and 120 minutes (compare the two lines of FIG. 5. This result is in agreement with the thermal stability measured by DSLS.

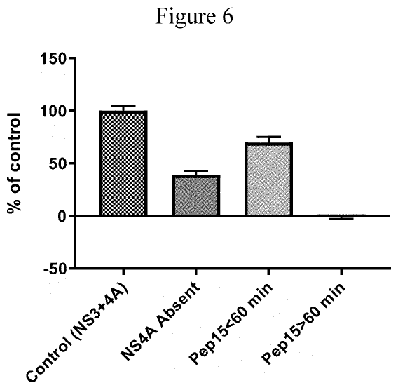

The competitive assay confirmed that the peptide of SEQ ID NO: 20 binds to the same binding as that of the labeled peptide of SEQ ID NO: 4, i.e., the activation site of SEQ ID NO: 3. The protease activity of NS3 of SEQ ID NO: 3 was examined in the presence and absence of the peptides of SEQ ID NO: 4 and 20 (see FIG. 6). The protease activity was measured using Sensolyte™ kit containing the Fam-peptide substrate of SEQID NO: 22. The activity was monitored by following the change in fluorescence at 520 nm with time using excitation wave length at 490 nm. The activity observed from a solution comprising NS3 of SEQ ID NO: 3 (6 μM) the peptide of SEQ ID NO: 4 (6 μM) and 5FAM-substrate of SEQ ID NO: 22 (200 μM) incubated for 60 min was considered 100% activity. A solution of NS3 of SEQ ID NO: 3 (6 μM) comprising varied concentration of Pep-15 of SEQ ID NO: 20 in the range of 50 μM to 97.6 nM was incubated for 15 minutes, and the reaction was initiated by adding 5FAM-substrate of SEQ ID NO: 22.

NS3 of SEQ ID NO: 3 (without NS4A) exhibited 39% activity. A solution of NS3 of SEQ ID NO: 3 containing Pep-15 of SEQ ID NO: 20 in a concentration range of 50 μM to 97.6 nM maintained 69.7% of the enzyme activity during the first 60 minutes (Fluorescence were measured every 10 min intervals). After 60 minutes, it was observed that the activity of the enzyme completely disappeared. The observed inhibition lag time was expected because binding of NS4A of SEQ ID NO: 2 as well as the synthetic peptide analogues thereof display lag time to observe their effect on the enzyme (45-60 minutes) and similar results was observed in the DSLS and the fluorescence experiments.

Pep-5 of SEQ ID NO: 10, Pep-10 of SEQ ID NO: 15 and Pep-15 of SEQ ID NO: 20 analogues of residues 21 to 33 of HCV NS4A of SEQ ID NO: 2 were developed as an inhibitor of activation of HVC NS3 protease of SEQ ID NO: 1 based on replacement of amino acids in positions corresponding to residues 29, 25 and 23 of SEQ ID NO: 2, respectively, by (S)-cyclohexylglycine (xG). Non-proteinogenic substituted peptides showed good and reproducible binding to the NS3 of SEQ ID NO: 3 with increased thermal stabilities of the peptide-NS3 protease of SEQ ID NO: 3 complex. The novel peptide GS(xG)VIVGRIVLSG (Pep-15 of SEQ ID NO: 20) displayed higher binding affinity towards HCV-NS3 than SEQ ID NO: 4 containing the required amino acid residue for the activation of the enzyme in a competition assay using fluorescence anisotropy technique. Pep-15 of SEQ ID NO: 20 was able to form a complex with NS3 protease of SEQ ID NO: 3 which was not able to cleave the enzyme substrate. The invention discloses peptides containing non-proteinogenic amino acid such as (S)-cyclohexylglycine which may be utilized as therapies against Hepacivirus and potentially against other pathogenic viruses from Flaviviridae family such as Dengue virus and Zika virus.

1: A peptide comprising:
an amino acid sequence having at least 60% sequence identity to Y₁GSX₁VX₂VGRX₃VLSGY₂ (SEQ ID NO: 5),analogs thereof, derivatives thereof, salts thereof and/or solvates thereof,
wherein at least one X₁, X₂, and X₃ is a non-proteinogenic amino acid,
wherein the peptide inhibits the protease activity of NS3 protease of hepatitis C virus of SEQ ID NO: 1 or a variant thereof having at least 60% sequence identity by binding to the binding site of the activation peptide NS4A of SEQ ID NO: 2 or variants thereof having amino acid sequence identity of at least 60% to SEQ ID NO: 2 or a fragment thereof,
wherein Y₁ and Y₂ are independently selected from the group consisting of hydrogen, one or more amino acid residues, an organic moiety comprising ionizable group, and a fluorescent moiety, and
wherein Y₁ and Y₂ are independently one or more selected from the group consisting of a charged amino acid residue, an organic moiety comprising an ionizable group, and a fluorescent moiety.

2: The peptide of claim 1, wherein the peptide has at least 80% amino acid sequence identity to SEQ ID NO: 5.

3: The peptide of claim 1, wherein the non-proteinogenic amino acid is (S)-cyclohexylglycine (cG).

4: The peptide of claim 2, wherein X₁, X₂, and X₃ are cG, I, and I, respectively.

5: The peptide of claim 2, wherein X₁, X₂, and X₃ are V, cG, and I, respectively.

6: The peptide of claim 2, wherein X₁, X₂, and X₃ are V, I, and cG, respectively.

7: The peptide of claim 2, wherein the peptide has at least 70% sequence identity to an amino acid sequence selected from the group consisting of SEQ ID NO: 10, SEQ ID NO: 15, and SEQ IDNO: 20.

8: The peptide of claim 7 is selected from the group consisting of SEQ ID NO: 10, SEQ ID NO: 15, and SEQ IDNO: 20.

9: A pharmaceutical composition comprising one or more of the peptides of claim 1.

10: The pharmaceutical composition of claim 9, wherein the composition further comprises one or more carriers and/or excipients.

11: The pharmaceutical composition of claim 9, wherein the composition further comprises one or more additional antiviral compounds.

12: The pharmaceutical composition of claim 9, wherein the variant peptide binds to an activation site of hepatitis C virus (HCV) NS3 protease.

13: The pharmaceutical composition of claim 9, wherein the peptide has antiviral activity against the HCV.

14: The pharmaceutical composition of claim 9, wherein the peptide has at least 70% sequence identity to an amino acid sequence selected from the group consisting of SEQ ID NO: 10, SEQ ID NO: 15, and SEQ IDNO: 20.

15: A method of treating a subject infected with HCV, comprising:
administering to the subject an effective amount of the pharmaceutical composition of claim 9.

16: A method of protecting a subject from getting infected with HCV comprising administering to the subject an effective amount of a pharmaceutical composition of claim 9.

17: A method of modifying a peptide selected from the group consisting of SEQ ID NO: 4, 9, 10, 14, 15, 19, and 20 to obtain a peptide or chemical compound having increased antiviral activity relative to the parent peptide, wherein the method comprises:
constructing a three dimensional model of a HCV NS3 having amino acid sequence identity of at least 90% to SEQ ID NO: 1 using the atomic coordinates of Protein Data Bank of accession number selected from the group consisting of 1NS3, 20B0, 208M, 2OBQ, and 20C 1
docking a peptide selected from the group consisting of SEQ ID NO: SEQ ID NO: 4, 9, 10, 14, 15, 19, and 20 to the three dimensional model from (a),
modifying the structure of the docketed peptide to enhance the interactions between the resulting compound and the activator binding domain of NS3 protease,
synthesizing a compound having the modified structure, and
measuring the binding of the compound to an HCV NS3 protease having at least 60 sequence identity to SEQ ID NO: 1,
wherein a peptide or compound having enhanced binding to the activation site relative to the parent peptide is identified as an antiviral compound.

18: The method of claim 17, wherein a fluorescence assay method is used to measure the binding of the peptide or the compound to an HCV NS3 protease having at least 60% sequence identity to SEQ ID NO: 1 of SEQ ID NO: 1.

19: The method of claim 17, wherein a surface plasmon resonance binding assay is used to measure the binding of the peptide or the compound to an HCV NS3 protease having at least 60% sequence identity to SEQ ID NO: 1.

20: The method of claim 17, wherein the identified peptide or compound contains a substitution of at least one amido nitrogen of at least one peptide bond with a CR1R2 wherein R1 and R2 are independently hydrogen, optionally substituted alkyl, optionally substituted cycloalkyl, optionally substituted aryl, optionally substituted heterocyclic, or optionally substituted heteroaryl.


SEQUENCE LISTING (25 sequences)

SEQ ID NO: 1 - protein, 181 aa, Hepatitis C virus
APITAYAQQTRGLFSTIVTSLTGRDTNENCGEVQVLSTATQSFLGTAVNGVMWTVYHGAGSKTISGPKGPVNQMYTNVDQDLVGWPSPPGVKSLTPCTCGASDLYLVTRHADVVPVRRRGDTRGALLSPRPISTLKGSSGGPLLCPMGHAAGLFRAAVCTRGVAKAVDFVPVESLETTMRS

SEQ ID NO: 2 - protein, 54 aa, Hepatitis C virus
STWVLVGGVLAALAAYCLSVGSVVIVGRVILSGQPAIIPDREVLYQQFDEMEEC

SEQ ID NO: 3 - protein, 200 aa, Artificial Sequence
  Description of Artificial Sequence Synthetic peptide
MASMTGGQQMGAPITAYAQQTRGLFSTIVTSLTGRDTNENCGEVQVLSTATQSFLGTAVNGVMWTVYHGAGSKTISGPKGPVNQMYTNVDQDLVGWPSPPGVKSLTPCTCGASDLYLVTRHADVVPVRRRGDTRGALLSPRPISTLKGSSGGPLLCPMGHAAGLFRAAVCTRGVAKAVDFVPVESLETTMRSGSHHHHHH

SEQ ID NO: 4 - protein, 17 aa, Artificial Sequence
  Description of Artificial Sequence Synthetic peptide
KKGSVVIVGRIVLSGKK

SEQ ID NO: 5 - protein, 15 aa, Artificial Sequence
  Description of Artificial Sequence Synthetic peptide
  MOD_RES (1)..(1): Any charged amino acid or absent
  MOD_RES (4)..(4): Any amino acid or any non-proteinogenic amino acid
  MOD_RES (6)..(6): Any amino acid or any non-proteinogenic amino acid
  MOD_RES (10)..(10): Any amino acid or any non-proteinogenic amino acid
  MOD_RES (15)..(15): Any charged amino acid or absent
XGSXVXVGRXVLSGX

SEQ ID NO: 6 - protein, 17 aa, Artificial Sequence
  Description of Artificial Sequence Synthetic peptide
KKGSVVIVGRFVLSGKK

SEQ ID NO: 7 - protein, 17 aa, Artificial Sequence
  Description of Artificial Sequence Synthetic peptide
KKGSVVIVGRWVLSGKK

SEQ ID NO: 8 - protein, 17 aa, Artificial Sequence
  Description of Artificial Sequence Synthetic peptide
KKGSVVIVGRAVLSGKK

SEQ ID NO: 9 - protein, 17 aa, Artificial Sequence
  Description of Artificial Sequence Synthetic peptide
  MOD_RES (11)..(11): (S)-homoisoleucine
KKGSVVIVGRIVLSGKK

SEQ ID NO: 10 - protein, 17 aa, Artificial Sequence
  Description of Artificial Sequence Synthetic peptide
  MOD_RES (11)..(11): (S)-cyclohexylglycine
KKGSVVIVGRGVLSGKK

SEQ ID NO: 11 - protein, 17 aa, Artificial Sequence
  Description of Artificial Sequence Synthetic peptide
KKGSVVFVGRIVLSGKK

SEQ ID NO: 12 - protein, 17 aa, Artificial Sequence
  Description of Artificial Sequence Synthetic peptide
KKGSVVWVGRIVLSGKK

SEQ ID NO: 13 - protein, 17 aa, Artificial Sequence
  Description of Artificial Sequence Synthetic peptide
KKGSVVAVGRIVLSGKK

SEQ ID NO: 14 - protein, 17 aa, Artificial Sequence
  Description of Artificial Sequence Synthetic peptide
  MOD_RES (7)..(7): (S)-homoisoleucine
KKGSVVIVGRIVLSGKK

SEQ ID NO: 15 - protein, 17 aa, Artificial Sequence
  Description of Artificial Sequence Synthetic peptide
  MOD_RES (7)..(7): (S)-cyclohexylglycine
KKGSVVGVGRIVLSGKK

SEQ ID NO: 16 - protein, 17 aa, Artificial Sequence
  Description of Artificial Sequence Synthetic peptide
KKGSFVIVGRIVLSGKK

SEQ ID NO: 17 - protein, 17 aa, Artificial Sequence
  Description of Artificial Sequence Synthetic peptide
KKGSWVIVGRIVLSGKK

SEQ ID NO: 18 - protein, 17 aa, Artificial Sequence
  Description of Artificial Sequence Synthetic peptide
KKGSAVIVGRIVLSGKK

SEQ ID NO: 19 - protein, 17 aa, Artificial Sequence
  Description of Artificial Sequence Synthetic peptide
  MOD_RES (5)..(5): (S)-homoisoleucine
KKGSIVIVGRIVLSGKK

SEQ ID NO: 20 - protein, 17 aa, Artificial Sequence
  Description of Artificial Sequence Synthetic peptide
  MOD_RES (5)..(5): (S)-cyclohexylglycine
KKGSGVIVGRIVLSGKK

SEQ ID NO: 21 - DNA, 612 bp, Artificial Sequence
  Description of Artificial Sequence Synthetic polynucleotide
catatggcgagcatgaccggtggccagcaaatgggtgcgccgatcaccgcgtacgcgcagcaaacccgtggcctgttcagcaccattgtgaccagcctgaccggtcgtgataccaacgagaactgcggcgaagtgcaggttctgagcaccgcgacccagagcttcctgggcaccgcggtgaacggcgttatgtggaccgtgtaccacggtgcgggcagcaagaccatcagcggtccgaaaggcccggttaaccagatgtataccaacgtggaccaggacctggttggttggccgagcccgccgggcgtgaagagcctgaccccgtgcacctgcggtgcgagcgacctgtatctggtgacccgtcacgcggatgtggttccggttcgtcgtcgtggtgacacccgtggcgcgctgctgagcccgcgtccgattagcaccctgaaaggtagcagcggtggcccgctgctgtgcccgatgggtcatgcggcgggcctgtttcgtgcggcggtttgcacccgtggtgtggcgaaagcggttgactttgtgccggttgagagcctggaaaccaccatgcgtagcggcagccaccaccaccaccaccactaaggatcc

SEQ ID NO: 22 - protein, 9 aa, Artificial Sequence
  Description of Artificial Sequence Synthetic peptide
SLGRKIQIQ

SEQ ID NO: 23 - DNA, 2055 bp, Hepatitis C virus
gcccccatcacggcgtacgcgcagcagacccgtggcttgttcagtaccatcgtaacgagcctcactggcagggacaccaatgaaaactgtggtgaagtacaggttctgtccaccgccacgcagtccttcctgggtactgcagtcaacggcgtgatgtggaccgtctaccacggggcgggtagcaagaccatcagcggcccaaagggacctgtcaatcaaatgtacaccaatgttgaccaagacctggtggggtggccatcgcctcccggagtcaaatctctcacaccatgcacctgtggcgcgtctgacttgtacttagtcaccaggcatgcagacgtggtgcccgtgcgcaggagaggggacaccagaggagctctcttgagccccaggcccatatccacacttaaggggtcttctggtggtccgctgctgtgccccatgggacacgctgccggcttgttccgcgcggcggtgtgcactcgaggggtggctaaagcggtggacttcgtcccggttgaatcccttgagactacaatgagatcaccagtgttcactgacaactcaacaccccctgcagtgccccagacctaccaggtcgcgcacttgcacgcgccaacaggtagtggcaagagcactaaagtcccggcagcgtacgcagctcagggctataaagtgctagtgctcaacccctcggttgctgccacactgggatttggggcgtacatgtccaaggcatacggcattgacccaaacattcggtcaggagtcaggactatcaccacgggtgcgccaatcacgtactccacgtatggtaagttcctggctgatgggggttgcagcgggggggcatacgacataatcatctgtgacgagtgccattccactgactccacgacgatccttggcataggcacggtcttggaccaagcggagactgctggagtgcgccttgtcgtgctcgcaaccgctaccccgccagggtcggtgactacaccccattccaacatagaggaggtcgccctgccaacgacgggggaaatacccttctacggcaaggcgattcccctagagctaatcaaggggggcagacacctcatcttttgtcactcaaagaagaagtgtgatgaactggctaaacaattaacatctctcggtctgaatgctgtagcctactacagaggcttagacgtttcggtgattcccacttctggggacgtcgtggtatgtgccacggacgccctcatgacggggttcaccggcgactttgactcggtgatagactgcaatacatctgtaatacagaccgtcgacttcagcttggaccccaccttctctatagagacaacaaccgttcctcaggatgcggtatcccgcagccagcggagaggccgcactggtagggggaggttgggtatataccggtatgtcaccccaggggagagaccatcaggcatattcgacacttcagtgatctgtgagtgctacgatgctgggtgcgcctggtatgagctgacaccggctgagaccacaacaaggctgagagcttacttcaatacaccgggccttcctgtgtgccaagaccacctggagttctgggagagcgtctttacagggttgacccaaatagacggccatttcctatcccagaccaagcaatcgggtgagaattttccataccttgttgcatatcaggccacagtatgcgccagagctctggcacccccaccaagctgggacaccatgtggaagtgcctaatccgcctcaagcctaccctgcatgggcccacacctctcctctacagattgggggccgtgcagaatgaagttacacttacccatcctattaccaaatacatcatggcatgcatgtcagctgacctcgaagtagtgacgagcacgtgggtcctagtgggcggcgtcctggcagccctagctgcttactgtctttcagtgggcagcgtggtgatcgtcgggagagttatcctgtcgggccaacctgctatcattcctgaccgcgaggtgctctaccaacagttcgacgaaatggaggagtgc

SEQ ID NO: 24 - protein, 685 aa, Hepatitis C virus
APITAYAQQTRGLFSTIVTSLTGRDTNENCGEVQVLSTATQSFLGTAVNGVMWTVYHGAGSKTISGPKGPVNQMYTNVDQDLVGWPSPPGVKSLTPCTCGASDLYLVTRHADVVPVRRRGDTRGALLSPRPISTLKGSSGGPLLCPMGHAAGLFRAAVCTRGVAKAVDFVPVESLETTMRSPVFTDNSTPPAVPQTYQVAHLHAPTGSGKSTKVPAAYAAQGYKVLVLNPSVAATLGFGAYMSKAYGIDPNIRSGVRTITTGAPITYSTYGKFLADGGCSGGAYDIIICDECHSTDSTTILGIGTVLDQAETAGVRLVVLATATPPGSVTTPHSNIEEVALPTTGEIPFYGKAIPLELIKGGRHLIFCHSKKKCDELAKQLTSLGLNAVAYYRGLDVSVIPTSGDVVVCATDALMTGFTGDFDSVIDCNTSVIQTVDFSLDPTFSIETTTVPQDAVSRSQRRGRTGRGRLGIYRYVTPGERPSGIFDTSVICECYDAGCAWYELTPAETTTRLRAYFNTPGLPVCQDHLEFWESVFTGLTQIDGHFLSQTKQSGENFPYLVAYQATVCARALAPPPSWDTMWKCLIRLKPTLHGPTPLLYRLGAVQNEVTLTHPITKYIMACMSADLEVVTSTWVLVGGVLAALAAYCLSVGSVVIVGRVILSGQPAIIPDREVLYQQFDEMEEC

SEQ ID NO: 25 - protein, 18 aa, Artificial Sequence
  Description of Artificial Sequence Synthetic peptide
  MOD_RES (1)..(1): 6-aminohexanoic acid
XKKGSVVIVGRIVLSGKK